(19) United States
(12) Patent Application Publication
(10) Pub. No.: US 2002/0168659 A1
(43) Pub. Date: Nov. 14, 2002

(54) System and method for characterizing the permittivity of molecular events

(75) Inventors: John Hefti, San Francisco, CA (US); Hong Peng, Fremont, CA (US); David Balaban, San Jose, CA (US); Joseph Heanue, Half Moon Bay, CA (US)

Correspondence Address: SIGNATURE BIOSCIENCE, INC., 475 Brannan Street, San Francisco, CA, 94107, US

(73) Assignee: Signature BioScience Inc., Hayward, CA

(21) Appl. No.: 10/073,827

(22) Filed: Feb. 11, 2002

Related U.S. Application Data
(60) Provisional application No. 60/268,401, filed on Feb. 12, 2001.
(60) Provisional application No. 60/275,022, filed on Mar. 12, 2001.

(51) Int. Cl. C12Q 1/68; G06G 7/48; G06G 7/58; G06F 19/00

(52) U.S. Cl. 435/6; 702/19; 703/11

(57) ABSTRACT

A system and method for comparing the permittivities of test and reference samples to detect and identify molecular events is presented. A resonant detector configured to output measurement parameters when the detector is electromagnetically coupled to a supplied sample is provided. One or more permittivity coefficients is defined for the detector. A first output parameter is obtained from the detector when the detector is electromagnetically coupled to a reference sample. A second output parameter is obtained from the detector when the detector is electromagnetically coupled to the test sample. The difference between the first and second output parameters is applied to the one or more permittivity coefficients to compute the relative difference in permittivity between the test sample and reference sample.

### BACKGROUND OF THE INVENTION

The present invention is related to the systems and methods for detecting and identifying molecular structures and binding events, and more particularly to systems and methods for monitoring the change in the permittivity of the test sample in which the molecular structure or binding event resides to detect and identify molecular structures and binding events.

Virtually every area of biological science is in need of a system to determine the ability of molecules of interest to interact with other molecules. Likewise, the ability to detect the presence and/or physical and functional properties of biological molecules on a small scale is highly desirable. Such molecular interactions, as well as the detection of functional and physical properties of molecules, are referred to here as “molecular events.”

Applicant's co-pending applications have disclosed systems and methods for detecting and identifying molecular events through the measurement of the dielectric properties of the sample under test. In particular, the applicant has described the use of scattering (or “s-”) parameters to quantitiate changes in the dielectric properties of the test sample as a means to detect and identify molecular events. In addition, the applicant has filed Atty Docket No. 16.0 US, entitled “System and Method for Detecting and Identifying Molecular Events in an Aqueous Sample using a Resonant Test Structure” in which the Applicant describes the use of the resonant frequency fᵣₑₛ and quality factor of a resonant probe to detect and identify molecular events in a test sample.

While the aformentioned parameters are useful in detecting and identifying molecular events, they are sample-volume dependent and test system specific. In particular, the measured s-parameters, the resonant frequency and quality factor of the resonant probe are strongly influenced by any variation in the volume of sample which can easly vary between different test systems. In addition, these parameters are also affected by variations in how the probe and sample are interfaced, which can vary widely across test systems. As a consequence, measurements made on different test platforms are not easily comparable.

What is therefor needed is a system and method for detecting and identifying molecular events using a system-independent quantity. By so doing, measurements made on different test systems indicating the presence (or absence) and identity of molecular events in a test sample can be easly compared.

### SUMMARY OF THE INVENTION

The present invention provides a system and method for detecting and identifying molecular events in a sample by monitoring the sample's change in characterizing a test sample in terms of the test sample's permittivity. The permittivity of the test sample is largely independent of the sample's volume, sample/probe interface, and variations in test set performance. Measurements made on different test systems indicating the presence (or absence) and identity of molecular events in a test sample can be easly compared.

In one embodiment of the invention, a method for comparing the permittivities of test and reference samples to detect and identify molecular events is presented. A resonant detector configured to output measurement parameters when the detector is electromagnetically coupled to a supplied sample is provided. One or more permittivity coefficients is defined for the detector. A first output parameter is obtained from the detector when the detector is electromagnetically coupled to a reference sample. A second output parameter is obtained from the detector when the detector is electromagnetically coupled to the test sample. The difference between the first and second output parameters is applied to the one or more permittivity coefficients to compute the relative difference in permittivity between the test sample and reference sample.

The nature and advantages of the present invention will be better understood with reference to the following drawings and detailed description.

### BRIEF DESCRIPTION OF THE DRAWINGS

FIG. 1 illustrates a test system configured to measure the permittivity of the test sample in accordance with one embodiment of the present invention.

FIG. 2A illustrates a simplified block diagram of a computer system shown in FIG. 1 in accordance with the present invention.

FIG. 2B illustrates the internal architecture of the computer system shown in FIG. 1.

FIG. 3A illustrates one embodiment of a resonant detector in accordance with the present invention.

FIG. 3B illustrates an exemplary return loss response obtained using the resonant detector illustrated in FIG. 3A.

FIG. 3C illustrates one embodiment of a non-resonant detector in accordance with one embodiment of the present invention.

FIG. 3D illustrates a second embodiment of a non-resonant detector in accordance with the present invention.

FIG. 4A illustrates a first embodiment of a detector assembly in accordance with the present invention.

FIG. 4B illustrates a second embodiment of the detector assembly in accordance with the present invention.

FIG. 5 illustrates a method for characterizing the permittivity of a molecular event in a test sample in accordance with one embodiment of the present invention.

FIG. 6 illustrates an exemplary embodiment in which two permittivity coefficients are defined for a resonant detector.

FIG. 7A illustrates an exemplary embodiment in which permittivity difference quantities Δ∈′_{cal} and Δ∈″_{cal} are applied to measurement parameters fᵣₑₛ and Q to compute the permittivity coefficients.

FIG. 7B illustrates an exemplary embodiment in which fᵣₑₛ and Q parameters are applied to the permittivity coefficients C′ and C″ to compute the test sample permittivity.

FIG. 8A illustrates an exemplary embodiment in which computed permittivity difference quantities Δ∈′_{cal} and Δ∈″_{cal} are applied to the resistance (r) and reactance (x) parameters to compute the permittivity coefficients.

FIG. 8B illustrates an examplary embodiment in which parameters r and x are applied to the permittivity coefficients C′ and C″ to compute the test sample permittivity.

FIG. 9A illustrates an exemplary embodiment in which computed permittivity difference quantities Δ∈′_{cal} and Δ∈″_{cal} are applied to real (I) and imaginary (Q) components of an s-parameter measurement to compute the permittivity coefficients.

FIG. 9B illustrates an exemplary embodiment in which the I and Q components of the measured s-parameters are applied to the permittivity coefficients C′ and C″ to compute the test sample permittivity.

FIG. 10 illustrates a method for characterizing the permittivity of a molecular event in a test sample using a bilinear calibration technique.

FIG. 11 illustrates an exemplary permittivity versus frequency versus temperature response for a test sample in accordance with the present invention.

FIG. 12A illustrates a process for determining the temperature-dependent permittivity of a test sample in accordance with one embodiment of the present invention.

FIG. 12B illustrates a process for measuring a plurality of temperature-dependent permittivities for the test sample at a corresponding pluralities of distinct temperature in accordance with one embodiment of the present invention.

FIG. 12C illustrates a process for selecting the temperature-dependent permittivity of the test sample which most closely correlates to the computed temperature-independent permittivity.

### DESCRIPTION OF SPECIFIC EMBODIMENTS

Index of Contents

I. Defintion of Terms

II. General Overview

III. Test System Architecture

IV. Detector Embodiments

V. Permittivity Characterization Processes

I. Definition of Terms

As used herein, the term “molecular binding event” (sometimes shortened to “binding event” or “binding”) refers to the interaction of a molecule of interest with another molecule. The term “molecular structure” refers to all structural properties of molecules of interest, including the presence of specific molecular substructures (such as alpha helix regions, beta sheets, immunoglobulin domains, and other types of molecular substructures), as well as how the molecule changes its overall physical structure via interaction with other molecules (such as by bending or folding motions), including the molecule's interaction with its own solvation shell while in solution. Together, “molecular structures” and “molecular binding events” are referred to as “molecular events.” The simple presence of a molecule of interest in the region where detection/analysis is taking place is not considered to be a “molecular event,” but is referred to as a “presence.”

Examples of molecular binding events are (1) simple, non-covalent binding, such as occurs between a ligand and its antiligand, and (2) temporary covalent bond formation, such as often occurs when an enzyme is reacting with its substrate. More specific examples of binding events of interest include, but are not limited to, ligand/receptor, antigen/antibody, enzyme/substrate, DNA/DNA, DNA/RNA, RNA/RNA, nucleic acid mismatches, complementary nucleic acids and nucleic acid/proteins. Binding events can occur as primary, secondary, or higher order binding events. A primary binding event is defined as a first molecule binding (specifically or non-specifically) to an entity of any type, whether an independent molecule or a material that is part of a first surface, typically a surface within the detection region, to form a first molecular interaction complex. A secondary binding event is defined as a second molecule binding (specifically or non-specifically) to the first molecular interaction complex. A tertiary binding event is defined as a third molecule binding (specifically or non-specifically) to the second molecular interaction complex, and so on for higher order binding events.

Examples of relevant molecular structures are the presence of a physical substructure (e.g., presence of an alpha helix, a beta sheet, a catalytic active site, a binding region, or a seven-trans-membrane protein structure in a molecule) or a structure relating to some functional capability (e.g., ability to function as an antibody, to transport a particular ligand, to function as an ion channel (or component thereof), or to function as a signal transducer).

Structural properties are typically detected by comparing the signal obtained from a molecule of unknown structure and/or function to the signal obtained from a molecule of known structure and/or function. Molecular binding events are typically detected by comparing the signal obtained from a sample containing one of the potential binding partners (or the signals from two individual samples, each containing one of the potential binding partners) to the signal obtained from a sample containing both potential binding partners. Together, the detection of a “molecular binding event” or “molecular structure” is often referred to as “molecular detection.”

The methodology and apparatuses described herein are primarily of interest to detect and predict molecular events of biological and pharmaceutical importance that occur in physiological situations (such as in a cellular or subcellular membrane or in the cytosol of a cell). Accordingly, structural properties of molecules or interactions of molecules with each other under conditions that are not identical or similar to physiological conditions are of less interest. For example, formation of a complex of individual molecules under non-physiological conditions, such as would be present in the vacuum field of an electron microscope, would not be considered to be a preferred “molecular binding event,” as this term is used herein. Here preferred molecular events and properties are those that exist under “physiological conditions,” such as would be present in a natural cellular or intercellular environment, or in an artificial environment, such as in an aqueous buffer, designed to mimic a physiological condition. It will be recognized that local physiological conditions vary from place to place within cells and organisms and that artificial conditions designed to mimic such conditions can also vary considerably. For example, a binding event may occur between a protein and a ligand in a subcellular compartment in the presence of helper proteins and small molecules that affect binding. Such conditions may differ greatly from the physiological conditions in serum, exemplified by the artificial medium referred to as “normal phosphate buffered saline” or PBS. Preferred conditions of the invention will typically be aqueous solutions at a minimum, although some amounts of organic solvents, such as DMSO, may be present to assist solubility of some components being tested. An “aqueous solution” contains at least 50 wt. % water, preferably at least 80 wt. % water, more preferably at least 90 wt. % water, even more preferably at least 95 wt. % water. Other conditions, such as osmolality, pH, temperature, and pressure, can and will vary considerably in order to mimic local conditions of the intracellular environment in which, for example, a binding event is taking place. The natural conditions in, for example, the cytosol of a cell and a lysosome of that cell, are quite different, and different artificial media would be used to mimic those conditions. Examples of artificial conditions designed to mimic natural ones for the study of various biological events and structures are replete in the literature. Many such artificial media are sold commercially, as exemplified by various scientific supply catalogues, such as the 2000/2001 issue of the Calbiochem General Catalogue, pages 81-82, which lists 60 commercially available buffers with pH values ranging from 3.73 to 9.24 typically used in biological investigations. Also see general references on the preparation of typical media, such as chapter 7 (“The Culture Environment”) of Culture of Animal Cells: A Manual of Basic Techniques, Third Edition, R. Ian Freshney, Wiley-Liss, N.Y. (1994).

As used herein, the term “electromagnetically coupled” will generally refer to the transfer of electromagnetic energy of between two or more structures. The term “directly coupled” will be used to describe the arrangment in which the structures (e.g., the sample and transmission line) come into direct contact and transfer electromagnetic energy between them. The term “indirectly coupled” will be used to describe this arrangement in which the structures are physically separated (e.g., through a matrix layer or barrier deposited along the transmission line, through the material which makes up a microfludic channel or PTFE flow tube, or through the aqueous environment of the molecular structure or binding event) but remain electromagnetically coupled to each other.

As used herein, the term “test signal” refers to an ac time varying signal. In specific embodiments, the test signal is preferably at or above 10 MHz (10×10⁶ Hz), such as 20 MHz, 45 MHz, 100 MHz, 250 MHz, 500 MHz, 750 MHz, 1 GHz (1×10⁹ Hz), 2 GHz, 5 GHz, 7.5 GHz, 10 GHz, 12 GHz, 15 GHz, 18 GHz, 20 GHz, 22 GHz, 25 GHz, 28 GHz, 30 GHz, 32 GHz, 40 GHz, 44 GHz, 50 GHz, 60 GHz, 110 GHz, 200 GHz, 500 GHz, 1000 GHz and range anywhere therebetween. A preferred region is from 10 MHz to 40 GHz, and more particularly from 45 MHz to 20 GHz.

As used herein, the terms “reference sample” or “test sample” refer to a buffer which contains, or is suspected to contain the biological or chemical molecular structures or binding event. As with biological samples, pretreatment of a more general sample (by dilution, extraction, etc.) once it is obtained from a source material does not prevent the material from being referred to as a sample. The buffer may consist of solid, liquid, or gaseous phase materials. Solid phase buffers may consist of conventional beads (non-magnetic or para-magnetic), naturally occurring or synthetic molecules including carbohydrates, proteins, oligonucleotides, SiO₂, GaAs, Au, or alternatively, any organic polymeric material, such as Nylon®, Rayon®, Dacryon®, polypropylene, polystryrene, Teflon®, Neoprene, Delrin or the like. Liquid phase buffers include those containing an aqueous, organic or other primary components, gels, gases, and emulsions. Exemplary buffers include celluloses, dextran derivatives, aqueous solution of d-PBS, Tris transporting media, deionized water, DMSO, blood, physiological transporting medium, cerebrospinal fluid, urine, saliva, water, organic solvents.

As used herein, the term “measurement parameter” refers to any conventional signal parameter representation, some examples being g-parameters, h-parameters, s-parameters, y-parameters, z-parameters. The term “electrical parameter” refers to any parameter which can be derived from any of the aforementioned measurement parameters, some examples being the resonant frequency fᵣₑₛ and quality factor (Q) associated with the response of a resonant structure, and the measured resistance and reactance of the detector (r and x). Collectively, measurement parameters and electrical parameters will be referred to as “output parameters.”

As used herein, the term “permittivity coefficient” refers to a quantity which represents the ratio between the measured permittivity of a sample and the output parameter of the detector at each measured frequency.

As used herein, the term “temperature-blind permittivity” refers to the sample permittivity which is computed computed without knowledge of the sample temperature.

As used herein, the term “temperature-informed permittivity” refers to the sample permittivity which is computed of a sample having a temperature dependency.

II. General Overview

As known in the art, the complex permittivity of a material at a given frequency (f) is given by:

∈(f)=∈′−j∈″ (1)

where:

∈′ is the real part of the permittivity and represents the dielectric constant of the material, and

∈″ is the imaginary part of the permittivity and represents the dielectric loss of the material.

As known to those skilled in the art of material properties, the dielectric constant represents a ratio of two parallel-plate capacitances of equal dimensions, one capacitance having the subject material interposed between its plates, and the second capacitance having a vacuum interposed between its plates. The dielectric loss of the sample represents energy dissipation of the material.

The real and imaginary parts of the permittivity changes over frequency, i.e., the sample will exhibit a a first dielectric constant ∈₁′ and dielectric loss ∈₁″ at one frequency and a different dielectric constant ∈₂′ and/or dielectric loss ∈₂″ at a second frequency. This variation over frequency (referred to as “dispersion”) results in a unique signal response when a test signal is electromagnetically coupled to the sample. The unique signal response enables the the sample is illuminated by a high frequency test signal. Provides the unique signal response for many samples, thereby enabling their detection and identification as described herein.

III. Test System Architecture

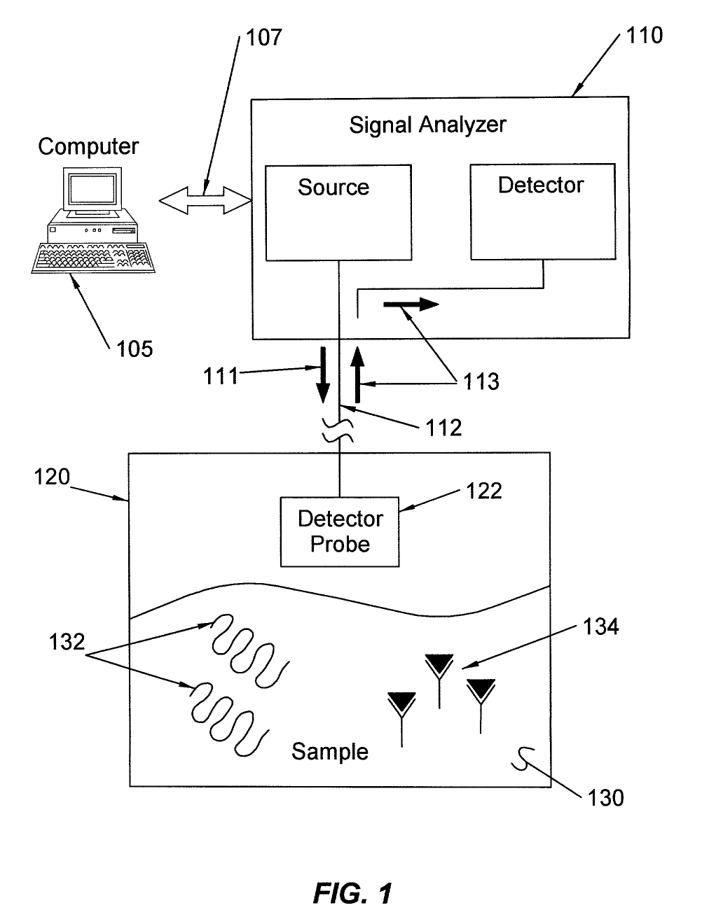

FIG. 1 illustrates a permittivity test set 100 configured to determine the permittivity of the test sample in accordance with one embodiment of the present invention. The test system 100 includes a computer 105, a signal analyzer 110, and a detector assembly 120. Computer 105 controls the settings and operation of signal analyzer 110 via a command bus 107 (a general purpose instrument bus in one embodiment). Responsive to the computer's instructions, signal analyzer 110 transmits an incident signal 111 along a signal path 112 (typically a coaxial cable) to the detector assembly 120. Within the detector assembly 120, a detector 122 is positioned proximate to the test sample 130, such that the detector 122 is electromagnetically coupled (either directly or indirectly, as defined above) to the test sample 130. In a specific embodiment, the test sample 130 is an aqueous environment which may contain molecular structures 132 and/or or binding events 134 as defined above.

As the incident signal 111 illuminates the test sample 130, the dielectric properties of the test sample 130 modulate the incident signal 111. At least a portion of the modulated incident signal is reflected back toward and is recovered by the coupling element 122. The incident and response signals 111 and 113 are subsequently analyzed to calculate the measured permittivity of the test sample 130.

Computer 105 may be any of a variety of commercially available computers such as series HP Vectra or HP 9000 available from the Hewlett Packard Company (Palo Alto, Cailf.). Others computation machines such as Macintosh or Unix-based machines may be employed in alternative embodiments. In a specific embodiment, computer 105 will include a graphical user interface such as Lab View provided by National Instruments (Austin, Tex.).

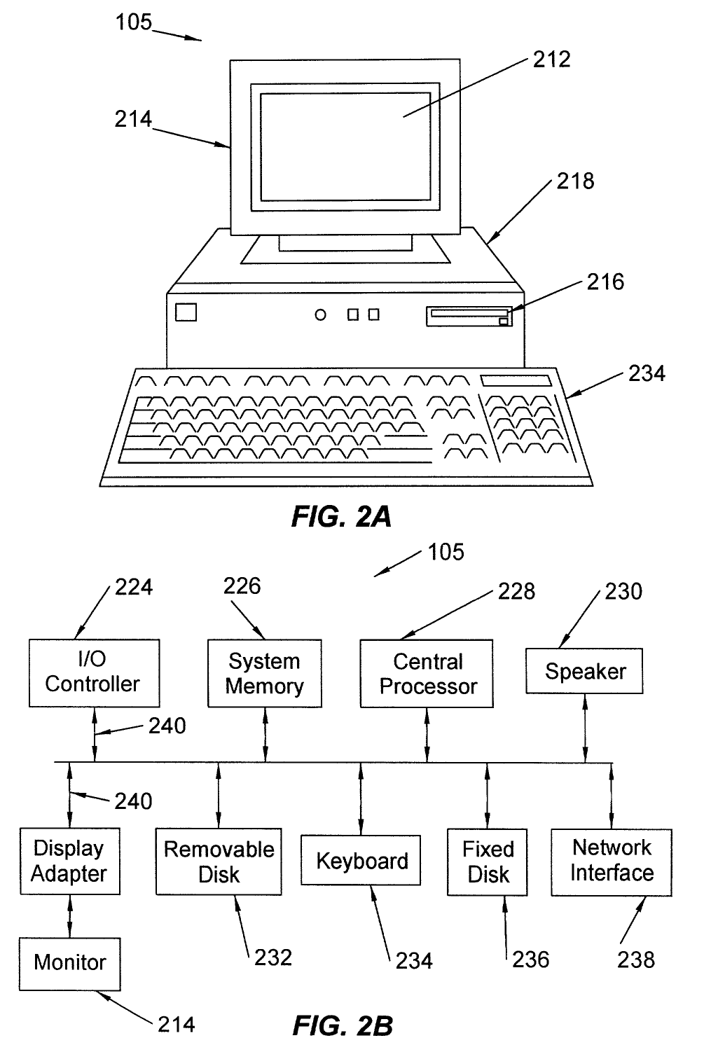

FIG. 2A illustrates a simplified block diagram of the computer system 105 operable to execute a software program designed to perform each of the methods described herein. The computer system 105 includes a monitor 214, screen 212, cabinet 218, and keyboard 234. A mouse (not shown), light pen, or other I/O interface, such as virtual reality interfaces can also be included for providing I/O commands. Cabinet 218 houses a CD/DVD-ROM/R/RW drive 216, a hard drive (not shown), or other storage data mediums which can be utilized to store and retrieve digital data and software programs incorporating the present method, and the like. Although drive 216 is shown as the removable media, other removable tangible media including floppy disks, tape, and flash memory can be utilized. Cabinet 218 also houses familiar computer components (not shown) such as a processor, memory, and the like.

FIG. 2B illustrates the internal architecture of the computer system 105. The computer system 210 includes monitor 214 which optionally is interactive with the I/O controller 224. Computer system 210 further includes subsystems such as system memory 226, central processor 228, speaker 230, removable disk 232, keyboard 234, fixed disk 236, and network interface 238. Other computer systems suitable for use with the described method can include additional or fewer subsystems. For example, another computer system could include more than one processor 228 (i.e., a multi-processor system) for processing the digital data. Arrows such as 240 represent the system bus architecture of computer system 210. However, these arrows 240 are illustrative of any interconnection scheme serving to link the subsystems. For example, a local bus could be utilized to connect the central processor 228 to the system memory 226. Computer system 210 shown in FIG. 2 is but an example of a computer system suitable for use with the present invention. Other configurations of subsystems suitable for use with the present invention will be readily apparent to of skill in the art.

Referring again to FIG. 1, signal analyzer 110 is operable to transit and receive the incident and response signals 111 and 113, respectively. Signal analyzer 110 may consist of any of a variety of commercially available instruments operable to transmit, receive, and measure the amplitude or amplitude and phase of signals. In a specific embodiment, signal analyzer 110 is a vector network analyzer model number 8722 manufactured by Agilent Technologies (formerly Hewlett Packard Company, Palo Alto; Calif.). In alternative embodiments, the signal analyzer may be a scalar network analyzer, a vector voltmeter, or other instrumentation capable of providing amplitude or amplitude and phase information of incident and reflected signals.

As illustrated in FIG. 1, two types of coupling elements are used in the preferred embodiment of the invention: a dielectric measurement probe 115 and a detector 120. The dielectric measurement probe 115 is used during the calibration process, further described below. In a specific embodiment, the dielectric measurement probe 115 is model no. HP 85070C dielectric measurement probe available from Agilent Technologies, Inc. (Palo Alto, Calif.). The detector assembly 120 is operable to measure the permittivity of the test sample, the process of which is also further explained below.

IV. Detector Embodiments

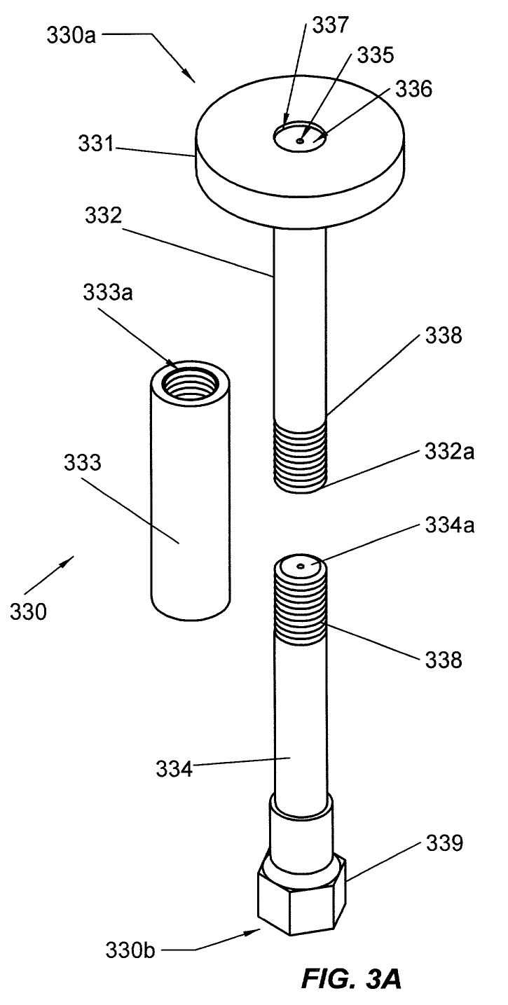

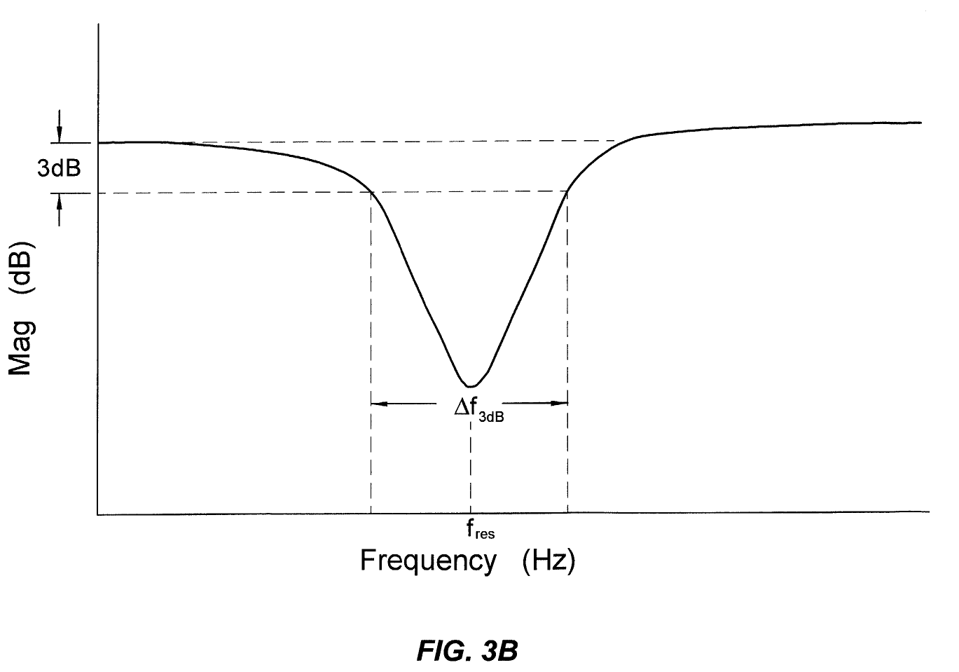

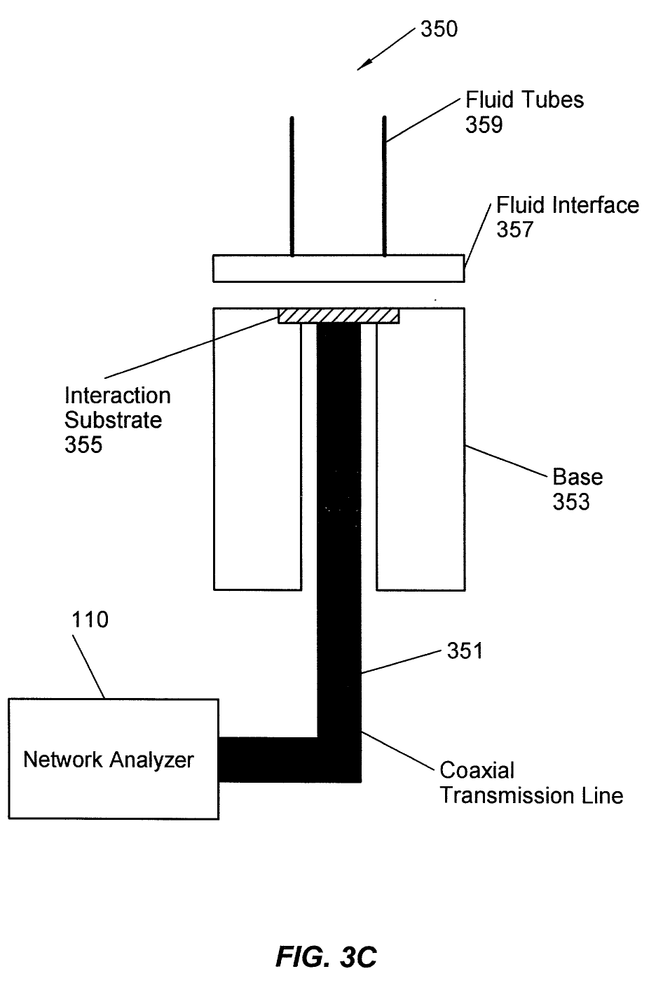

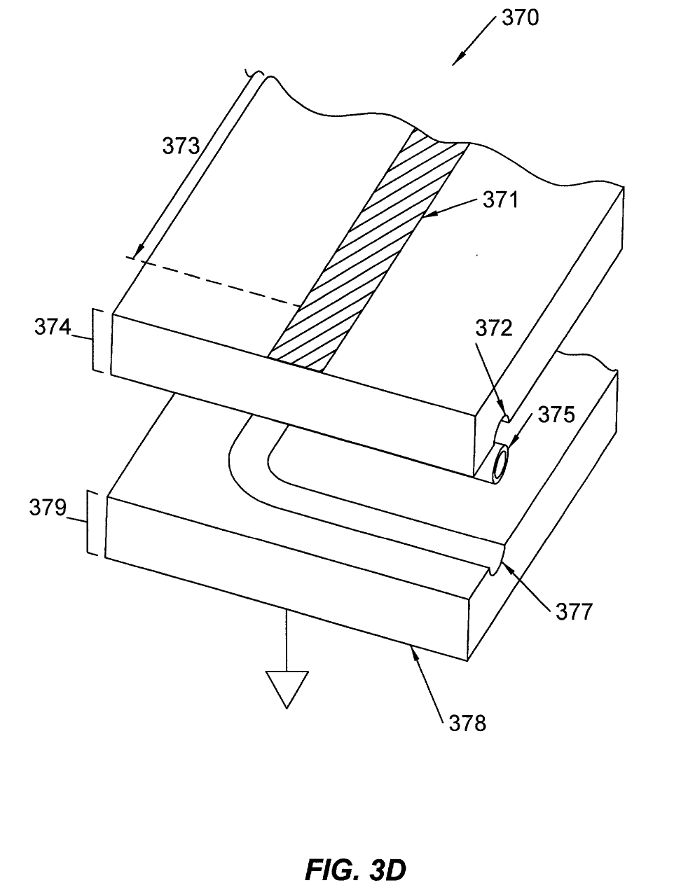

FIG. 3A illustrates one embodiment of a resonant detector 430 used to determine the permittivity of a test sample in accordance with the present invention. A specific embodiment of the detector is described in greater detail in applicant's commonly-owned, co-pending patent application Ser. No. 09/687,456 entitled: “System and Method for Detecting and Identifying Molecular Events in a Test Sample.”

As shown in FIG. 3A, the resonant detector 330 has two ports: a probe head 330a and a connecting end 330b. In a specific embodiment, the probe head 330a is an open-end coaxial cross section and the connecting end 330b is a coaxial-type connector, one embodiment of which is a SMA connector. Those of skill in the art will appreciate that other terminations (such as shorted or load terminations), as well as other circuit architectures (such as microstrip, stripline, coplanar waveguide, slot line, waveguide, etc.) can be used in alternative embodiments of the resonant detector 330.

The resonant detector 330 further includes two coaxial sections 332 and 334, each having a center conductor 335, a dielectric insulator 336 (air in a specific embodiment), and an outer conductor 337 (typically used to provide a ground potential reference). The first section 332 consists of the aforementioned probe head 330a and a first gap end 332a located opposite thereto, each realized as an open-end cross section of the coaxial cable. A shelf (preferably conductive) 331 is attached flush (preferably via solder, conductive epoxy or other conductive attachment means) with the outer conductor 337 of the probe head 330a.

The second section 334 is of similar construction as the first section 332, having a dielectric insulator 336 located between center and outer conductors 335 and 337. The second section 334 further includes a second gap end 334a and a connecting end 330b located opposite thereto. The second gap end is realized as an open-end cross section of the coaxial cable. The connecting end 330b is realized as a connector (SMA-type in a specific embodiment) operable to connect to the molecular detection system, further illustrated and described below. In a specific embodiment, the first and second sections 332 and 332 are each of the same dimensions as RG401 type semi-rigid coaxial cable, although larger or smaller diameter cables can be used as well The length of the first section 332 is calculated to be approximately one-half wavelength in length at the desired frequency of resonance.

In a specific embodiment of the invention, the resoant probe 330 includes a tuning element 333 which is adjustably engaged between the first and second gap ends 332a and 334a to provide a variable gap distance therebetween. The gap provides a capacitive effect between the first and second sections 332 and 334, and it, in combination with the electrical length of the first section 332, is designed to provide a resonant signal response when the probe 330 illuminates the test sample. The tuning element 333 can be rotated to expand or contract the gap (and according, decreasing or increasing the value of the capacitive effect) between the first and second sections 332 and 334, thereby changing the resonant frequency of the detector 330 to the desired frequency.

The tuning element 333 is preferably a hollow tube constructed from a material (stainless steel in one embodiment) that exhibits relatively high conductively to maintain ground potential between the first and second sections at the test frequency of operation. Further, the tuning element can include internal threads 333a which mate with external threads 338 disposed on the outer conductors of the first and second sections near the first and second gap ends 332a and 334a. In alternative embodiments of the invention, the tuning element 333 can be omitted, in which case the first and second sections 332 and 334 can comprise one continuous coaxial transmission line structure. The design of the resonant probe 330 is described in greater detail in applicant's co-pending patent applicant Ser. No. 09/687,456 entitled: “System and Method for Detecting and Identifying Molecular Events in a Test Sample.”

FIG. 3B illustrates an exemplary return loss response (referred to as a S₁₁) obtained using the resonant coaxial fixture 330. The response is characterized by an amplitude response (y-axis) extending over one or more frequencies (x-axis). As illustrated, the response exhibits a minimum amplitude at a frequency fᵣₑₛ typically referred to as the resonant frequency of the resonant probe. At this frequency, signal power will be substantially retained within the resonant probe. This is the frequency at which the probe is most sensitive since little power is dissipated within the probe itself. A parameter referred to as the “Quality” or “Q”-factor is used to measure how well the probe (or any resonant structure) retains signal power at its resonant frequency. Generally, the Q-factor is a ratio of the energy stored versus the energy dissipated at the resonant frequency fᵣₑₛ. Mathematically, the Q-factor can be expressed as:

Q=fᵣₑₛ/Δf_{3dB} (2)

where:

fᵣₑₛ is the resonant frequency at which the S11 amplitude is minimum; and

Δf−_{3dB} is the −3dB or half power bandwidth of the resonant detector above and below fᵣₑₛ

As can be seen in FIG. 3B, the smaller the half-power bandwidth around the resonant frequency point fᵣₑₛ, the higher the quality (i.e., lower dissipative loss and greater sensitivity) of the resonator. In a specific embodiment, the resonant probe of the present invention exhibits a fᵣₑₛ between 1 GHz and 1.5 GHz and a Q-factor of at least 200.

Those of skill in the art of high frequency circuit design will appreciate that the invention is not limited to the implementation of the illustrated detectors. Other resonant structures, such as cavities, filters, parallel or series resonant lumped element or distributed circuits, are but a few of the structures that could be used in alternative embodiments under the invention.

Those skilled in the art of high frequency design will appreciate that illustrating a response in the form of s-parameters is useful in displaying the response a wide range of magnitudes. For example in FIG. 3B, the amplitude of the resonant response (S₁₁) is displayed from 0 to −60 dB, thus illustrating the magnitude of the signal response over six orders of magnitude.

However, in some instances (e.g., where a small portion of the spectrum is being analyzed) it may be more convenient to display the response linearly. In such an instance, the response may be represented in terms of linear measurement parameters, y-(admittance) or z-(impedance) parameters being some examples.

The translation between these parameters is well-known in circuit analysis. For instance, when converting an input s-parameter (real and imaginary parts) to a z-parameter (resistance and reactance), the translation equation is given by:

z(r,x)=[1+(S₁₁{re,im})]/[1−(S₁₁{re,im})] (3)

where:

the impedance z(r,x) is normalized to the system's characteristic impedance (which is set to 1);

z(r,x)=complex impedance including resistance (r) and reactance (x) of the impedance z measured at each test frequency (f);

S11 {re,im,f}=complex return loss including real and imaginary parts measured at each test frequency (f).

As will be further described below, the permittivity coefficients may be described in terms of the linear electrical parameters, such as the z-parameters described above.

In a second embodiment of the invention, a non-resonant structure is used to determine the permittivity of a test sample. A non-resonant structure provides advantages in that it can be used to interrogate the test sample over a broad frequency spectrum, resulting in a broadband response obtained for the test sample. Because the molecular structures or binding events will typically exhibit dramatic and unique changes in the measured response at various frequencies over the broad frequency range, the test sample will have exhibit a unique response which can be used to identify molecular structures and binding events in subsequently tested samples.

FIG. 3C illustrates one embodiment of a non-resonant detector 350, realized as an open-ended coaxial probe (hereinafter referred to as “non-resonant probe”). The non-resonant probe 350 includes a section of open-ended coaxial line 351, an interaction fixture base 353, an interaction substrate 355, a fluid interface 357 having one or more fluid tubes 359 extending therefrom. In a specific embodiment, the probe 350 is coupled to a network analyzer or similar test equipment capable of measuring incident and reflected signal properties.

Fluid tubes 359 allows the introduction of sample into the fluid interface 357. An interaction substrate 355 may optionally be used to separate the supplied sample from the end of the coaxial section 351. The interaction substrate 355 may consist or a variety of materials, for example glass, quartz, polyimide, PTFE, materials such as silicon dioxide, gallium arsenide or other materials used in semiconductor processing. In alternative embodiments, interaction substrate 355 is removed and the sample comes into direct contact with the coaxial section 351. The base fixture 353 is used to securely attach and align the fluid interface 357 (and interaction substrate 355, if used) with the open end portion of the coaxial section 351. In a specific embodiment the base fixture 353 is aluminum, although other materials may be used in alternative embodiments of the present invention. The non-resonant probe 350 is described in further detail in applicant's commonly owned, co-pending U.S. patent application Ser. No. 09/687,456, entitled “System and Method For Detecting Molecular Events in a Test Sample.”

FIG. 3D illustrates the front portion of a microstrip detector 370 used to determine the permittivity of a test sample in accordance with one embodiment of the present invention (the rear portion is the mirror image of the front view). The microstrip detector 370 is described in further detail in applicant's commonly-owned, concurrently filed patent application entitled “Bioassay Device for Detecting Molecular Structures and Binding Events,” (Atty Dkt No. 15.0 US).

The microstrip detector 370 includes top and bottom dielectric plates 374 and 379 and a flow tube interposed therebetween. Top and bottom dielectric plates 374 and 379 are preferably constructed from a material exhibiting a low loss tangent at the desired frequency of operation. Suitable materials include alumina, glass, quartz, sapphire, beryllium, diamond, PTFE or variations thereof, materials used in semiconductor processing such as silicon dioxide and gallium arsenide, woven dielectric materials such as Rodgers Duriod® or other similar materials. In the illustrated embodiment, the dielectric plates 374 and 379 are each 0.030″ thick of GML 1000 (manufactured by Gil Technologies of Collierville, Tenn.) having a relative dielectric constant of approximately 3.2. While the dielectric plates 374 and 379 are of the same thickness and relative dielectric constant, variation in one or both of these may be used in alternative embodiments.

The top dielectric plate 374 includes a transmission line 371 deposited on the top surface and a channel 372 formed on the bottom surface. The width of transmission line 371 is selected to provide a predetermined characteristic impedance calculable from the dielectric constant and thickness of the top and bottom dielectric plates 374 and 379. The calculation may take into account the varying dielectric constants and dimensions introduced by channels 372 and 377 and flow tube 375. Alternatively, these features may be ignored and continuous dielectric plates assumed. The transmission line 371 may consist of any material which exhibits high conductivity of the desired test frequency(ies). Such materials include gold, copper, silver, indium tin oxide, or other similar metals. In the illustrated embodiment, the transmission line consists of 1 ounce copper.

The second dielectric plate 379 includes a channel 377 formed on the top surface and metallization deposited on the bottom surface. The channel 377 is aligned with channel 372 to form a cavity within which the flow tube 375 extends. The metallization 522 deposited on the bottom surface functions as the ground plane of the microstrip detector and will typically consist of a highly conductive material such as those described above. In an alternative embodiment, ground plane metallization may be deposited on the top surface of the top dielectric plate 374 to form a coplanar waveguide structure. Those of skill in the art of high frequency circuit design will appreciate that other configurations are also possible. In the illustrated embodiment, 0.002″ of copper is used as the bottom surface metallization to provide the detector's ground plane.

As shown in FIG. 3D, channels 372 and 377 are aligned to form a cavity which retains the flow tube 375 in a substantially vertically aligned position between the transmission line 371 and the ground plane 522. The flow tube is held between the transmission line 371 and the ground plane 522 along a longitudinally distance 373 referred to as the detection region. This configuration results in the passage of a significant number of field lines emanating from the transmission line through the flow tube (and accordingly, the test sample) before terminating on the ground plane 522. As discussed previously in this and the related applications, the dielectric properties of the sample flowing through the detection region will modulate the signal propagating along the transmission line 371 (i.e., by altering the field lines setup between the transmission line 371 and ground plane 522), thereby providing a means to detect and identify analytes or binding events occurring in the test sample.

In one embodiment, the microstrip detector includes connectors (not shown) connected to the transmission line 371 and ground plane 522 on the near and far sides. Suitable connectors are selected based upon the desired test frequency, N-type connectors being suitable for low frequency tests (<100 MHz), and SMA, K-type, 3.5 mm or 2.4 mm connectors being more suitable for higher frequency tests. Connection by other means, such as coplanar waveguide probes, may also be used in alternative embodiments.

In a specific embodiment, the microstrip detector is used as a one-port device with the far side connector terminated in an load closely matched to the characteristic impedance of the transmission line 371 in order to minimize reflections created by the termination. In this embodiment, portions of the modulated signal will be reflected back toward the signal source (coupled to the near side connector, not shown) and are detectable through a directional coupler. Those of skill in the art will understand that other arrangements are possible, for instance, using a highly reflective termination, or using the microstrip detector as a two port to determine the insertion loss response.

The flow tube 375 supplies the test sample through the detection region along 373 between the transmission line 371 and ground plane 522. In the preferred embodiment, the flow tube 375 is constructed from a material having a low loss tangent and a smooth, resilient surface morphology which inhibits analyte formation along the inner surface. A PTFE tube having an ID of 0.015″ and OD of 0.030″ is used in the illustrated embodiment, although other materials and/or sizes may be used as well. For example, materials such as ETFE or other materials described in this and the related cases may be used in alternative embodiments. Further, the flow tube 375 may consist of a microfluidic capillary such as those discussed in applicant's commonly owned, co-pending U.S. patent application Ser. No. 09/687,456, entitled “System and Method For Detecting Molecular Events in a Test Sample.” Applicant's commonly-owned, concurrently filed patent application entitled “Bioassay Device for Detecting Molecular Structures and Binding Events,” (Atty Dkt No. 15.0 US) further describes other non-resonant bioassay detectors, each of which may be similarly used in the aforementioned process.

Those of skill in the art of high frequency circuit design will appreciate that the invention is not limited to the implementation of the illustrated detectors. Any non-resonant, broadband, passive or active structures can be used to characterize the permittivity of molecular events within a sample using the methods of the present invention.

Detector Assembly Embodiments

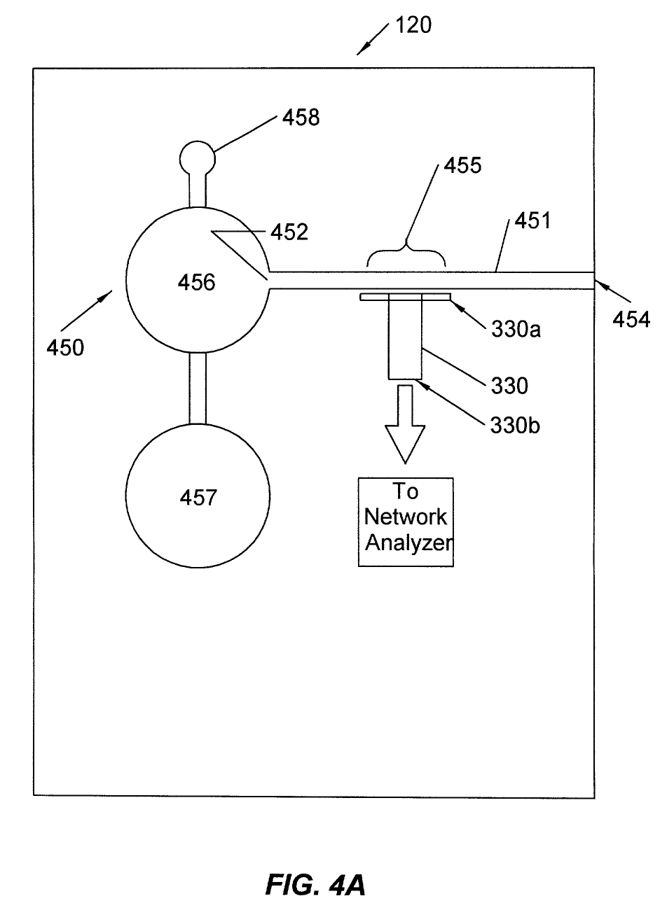

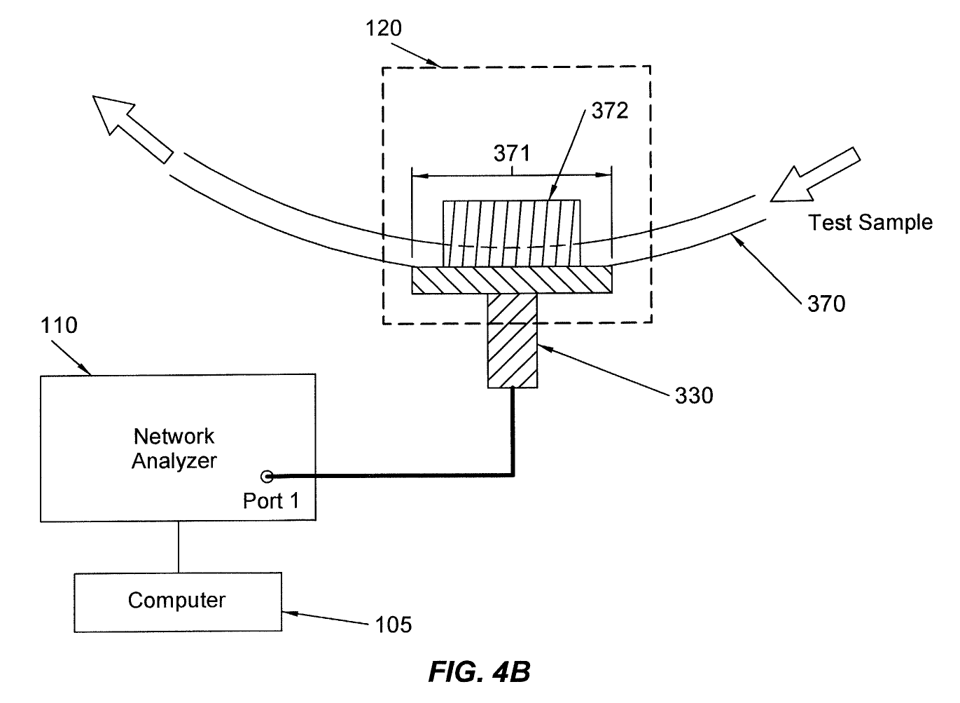

FIG. 4A illustrates one embodiment of the detector assembly 120 used to determine the permittivity of a test sample in accordance with the present invention. In this embodiment, the detector assembly 120 includes a fluid transport system 350 integrated with a detector 330. Embodiments of the detector assembly 120 is described in greater detail in applicant's commonly owned, co-pending patent application Ser. No. 09/678,456 entitled “System and Method for Detecting and Identifying Molecular Events in a Test Sample.”

The sample transport system 350 includes a fluid channel 351, with a entry end 352 and an exit end 354. Motion of the test sample through the channel 351 is controlled by a fluid controller 356, which acts to move the test sample through the channel at times and under conditions selected by the user. Optionally, reservoir 358 can include a second analyte or test sample that can be mixed with the test sample stored in reservoir 357 as they are being introduced to the fluid channel 351. The ability to mix two test samples in close proximity to the detector makes it easy for the kinetics of binding events to be determined from this type of data. The fluid controller 356 can move the test sample in one direction, in forward and reverse directions, or pause the test sample for a predetermined duration, for instance, over the detection region in order to improve sensitivity.

The detector assembly 330 includes probe head 330a and connecting end 330b. The probe head 330a is positioned proximate to the detection region 355 of the fluid channel 350 and is operable to electromagnetically couple (directly or indirectly, as defined above) the incident test signal to the test sample flowing through the detection region 355. The test sample modulates the incident signal, a portion of which is reflected to the probe head 330a. The reflected modulated signal is subsequently recovered by the probe head 330a. The connecting end 330b is electrically connected (directly or via intervening circuitry) to the signal analyzer 110. In a specific embodiment in which the detector is a coaxial-type structure, the connecting end 330b can be a coaxial cable which extends from the signal analyzer, a compatible coaxial type connector such as a SMA-type connector, or other connector type familiar to those skilled in the art of high frequency measurement. In alternative embodiments of the invention in which a different type of probe architecture is used (i.e., coplanar waveguide, microstrip, etc.), the connection port can comprise a compatible connection to provide signal communication to the permittivity test set.

FIG. 4B illustrates a second embodiment of the detector assembly 120. In this embodiment, the detector 120 includes an assembly of a length of RF permeable tubing 370, one example being PTFE type-tube available from Cole-Partner Instrument Company (Vernon Hills, Ill.). The tubing 370 transports the test sample to the detection region 371 illuminated by the detector 330. A cover piece 372, which is preferably constructed from a conductive material, includes a grooved portion through which tubing 370 extends.

In the illustrated embodiments of FIGS. 4A and 4B, the probe head 330 is indirectly coupled (as defined above) to the test sample by closely positioning the probe head proximate to the test sample. The intervening material(s) that physically separates the probe head 330a from the test sample can include solid phase materials, such as PTFE, alumina, glass, sapphire, diamond, Lexan®, polyimide, or other dielectric materials used in the area of high frequency circuit design; materials used in the fabrication of microfluidic devices or semiconductor processing; or other known materials which exhibit a relatively high degree of signal transparency at the desired frequency of operation. In a specific embodiment, the intervening material can be an electrically insulating material, some examples of which are described above. In embodiments in which the outer wall of a tube is the intervening material, the material may also be visually translucent or transparent to permit visual inspection of the sample as it moves through the tube. In specific embodiments, the tube may be made from fluoropolymers, such as PFA (perfluoro alkoxy alkane), PTFE (poly-tetra-fluoro-ethylene), FEP (fluorinated ethylene propylene), or ETFE (ethylene-tetrafluroethylene, copolymer), to name a few. The flow cell (not shown) may be constructed from a variety of materials such as (poly) methyl methacrylate—PMMA—acrylic, polycarbonate (known as Lexan®), or polyetherimide (known as Ultem®), as well as others.

Alternatively or in addition, liquid and/or gaseous phase materials (including air) that exhibit a relatively high degree of test signal transparency can also comprise the intervening materials.

The thickness and dielectric properties of the intervening materials can vary depending upon the type of fluidic system implemented and detector used. For instance, in systems in which the separation distance is great, a low loss, high dielectric material is preferred to provide maximum coupling between the test sample and the probe 330. In systems in which the separation distance is relatively short, materials of higher loss and lower dielectric constant can be tolerated. In a specific embodiment in which the channel 151 is PTFE tube having dimensions of 0.031 inch I. D., 0.063 inch O.D., wall thickness 0.016 inch, and a dielectric constant of approximately 2, the separation distance is approximately the tube's wall thickness, about 0.016 inch. In other detector assemblies, separation distances can be on the order of 10⁻¹ m, 10⁻² m, 10⁻³ m, 10⁻⁴ m, 10⁻⁵ m, or 10⁻⁶ m, and can be much smaller, e.g., on the order of 10⁻⁹ m in some cases (such as in a channel etched into the surface of a substrate and having a metallic signal path element with a thin polymer layer on the test sample side acting as the fourth side of the channel). Decreasing the separation distance or increasing the detection area 155, the sample volume, or analyte concentration will operate to increase detection sensitivity. The separation material, as illustrated above, can a solid phase material, or alternatively (or in addition) consist of a liquid or gaseous phase material or a combination thereof.

In an alternative embodiment, the probe head 330a and test sample may be directly coupled (as defined above), in which case the test sample comes into direct contact with the probe head 330a. In this embodiment, measurement sensitivity is increased as the signal loss contributed by the intervening material is not present. This embodiment may be realized in a variety of ways, for instance in FIG. 4A by extending the center conductor 335 such that it contacts the test sample moving through the detector region 355 of the fluid channel 351. In such an embodiment, the channel substrate (the material on which the fluid channel 351 is formed) may include a cavity within the detector region 351 for receiving the center conductor 337. The dielectric properties of the channel substrate may be used and the outer conductor of the detector extended to maintain the characteristic impedance of the detector (typically 50 ohms). Alternative realizations in which the test sample contacts the probe head 330a in the illustrated and alternative embodiments will be readily apparent to those skilled in the art.

V. Permittivity Characterization Processes

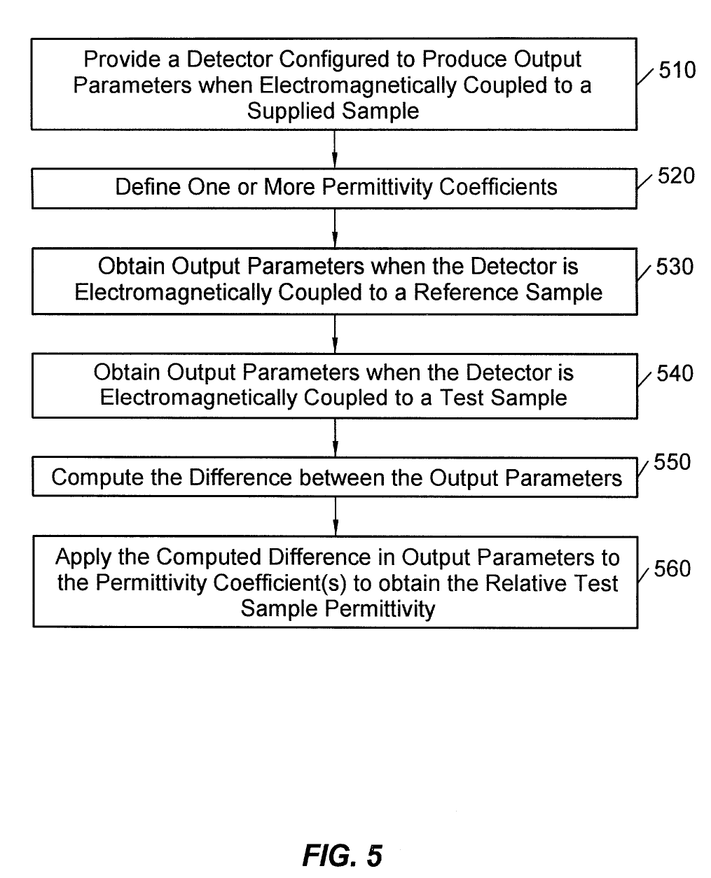

FIG. 5 illustrates a first method for characterizing the permittivity of a molecular event in a test sample in accordance with one embodiment of the present invention. In the illustrated example, the permittivity characterization is made relative to the permittivity of a reference sample. The reference sample may consist of a variety of different compositions, for instance, the reference sample may consist of the buffer component of the test sample, a ligand having a known affinity to bind to an antiligand suspected of being contained within the test sample, the binding complex between the ligand and antiligand, or other compositions. In another embodiment, the permittivity characterization provides an absolute value of the molecular event's permittivity.

At 510 a detector is provided, the detector being operable to produce output parameters when the detector is electromagnetically coupled to a supplied sample. The detector's output parameters may consist of conventional circuit measurement parameters such as g-, h-, s-, y-, or z-parameters. Alternatively, other quantities, such as the resonant frequency (fᵣₑₛ) and quality factor (Q), which can be derived from these parameters may also be used, as will be illustrated below.

As illustrated in FIGS. 3A, 3C and 3D, the detector may be of resonant or non-resonant architecture and have one or multiple signal input and output ports. The detector may form part of an integrated unit (as in FIG. 4A), an assembly (as in FIG. 4B), or comprise a separate item which is connected to the signal analyzer (as in FIG. 3C). The method of the present invention is not limited to the use of the illustrated detectors, and other resonant and non-resonant structures such as cavities, filters, parallel or series resonant lumped element or distributed circuits (active or passive), are but a few of the structures that could be used in alternative embodiments under the invention.

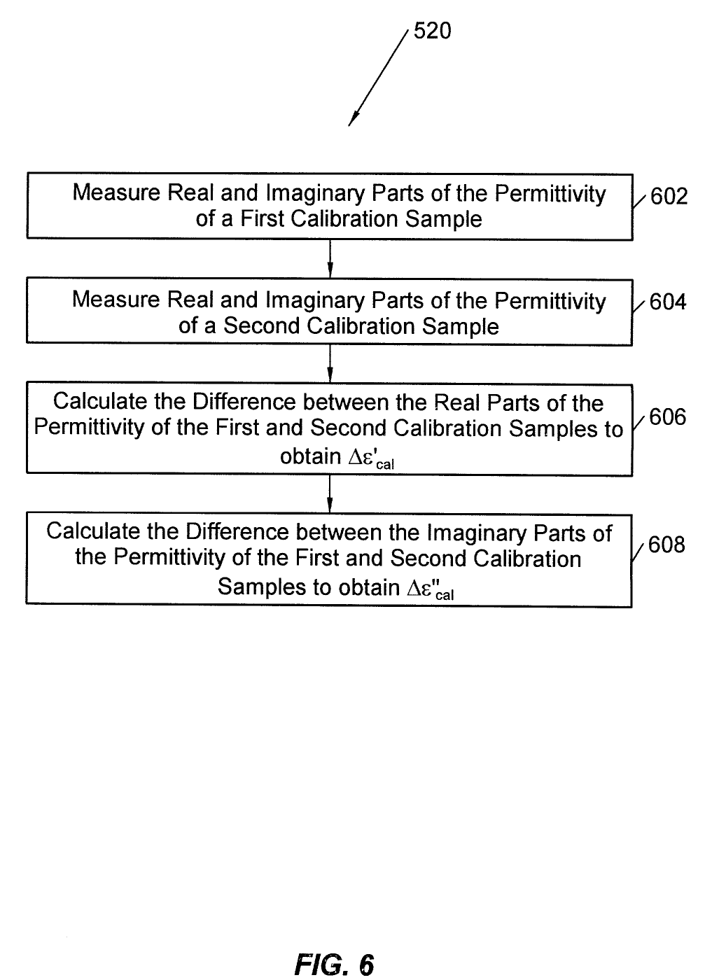

At 520, one or more permittivity coefficients are defined for the selected detector. In a specific embodiment, two permittivity coefficients are defined: a real coefficient C′ and a imaginary coefficient C″. In an alternative embodiment, only one permittivity coefficient is calculated. In still a further embodiment, three or more coefficients may be computed. For instance, a third coefficient indicating the degree of correlation between the real and imaginary parts of the electrical parameter (typically assumed to be zero) may be used. The computation of the permittivity coefficients are illustrated in greater detail in FIGS. 6A-C below.

At 530, the detector's output parameters are obtained while the detector is electromagnetically coupled to a reference sample. The reference sample is the material against which the test sample will be compared during the measurement process. The reference sample may be a sample of the native or near-native environment in which the sought molecular event is known to reside, e.g., a sample containing a purified protein, a mixture of proteins, complex systems such as cellular lysates.

The output parameters, in one embodiment, consist of a set of s-parameters, each s-parameter taken at a specific frequency and consisting of the ratio of an output signal to the incident test signal. As an exemplary embodiment, the reference sample is first supplied to the detection region 455 of the one-port detector 120. Next, an incident test signal 111 is launched from the test set 110, along the signal path 112, toward the detector probe head 330a. The incident signal 111 illuminates the sample and molecular event within the detection region and a response signal 113 reflected back towards the test set is recovered by the probe head 330a. The response signal 112 is recovered by the test set 110 which computes the resulting s-parameter consisting of the ratio of the response signal 112 (in amplitude and phase) to the incident signal 111 (amplitude and phase). The process may be repeated at additional frequencies to provide an s-parameter response over a spectrum of different frequencies. In another embodiment in which a resonant detector is employed, the measurement parameters consist of the resonant frequency fᵣₑₛ and quality factor Q of the detector. This and additional embodiments are described below.

At 540, the detector's output parameters are obtained while the detector is electromagnetically coupled to a test sample. This process may be performed in a manner similar to that illustrated in 530 above. Preferably, the same type of output parameters are obtained for processes of 530 and 540, for instance input s-parameters.

At 550, an output parameter difference quantity ΔM representing the difference between the output parameters obtained in processes 530 and 540, is computed. In one embodiment, the output parameter difference quantity (ΔM) is the difference in input s-parameters obtained from processes 530 and 540. In another embodiment, the output parameter difference quantity ΔM is the difference in the resonant frequencies and/or quality factors obtained from processes 530 and 540. These and other embodiments are described below.

At 560, a relative test sample permittivity value Δ∈ is computed by applying the defined permittivity coefficient(s) C to the output parameter difference quantity ΔO:

eqΔO×C=Δ∈ (4)

The resulting permittivity quantity represents the permittivity of the test sample relative to the reference sample and is substantially independent of the test system and detector used to obtain the measurement. Once this quantity is associated with the test sample, this value may then be stored in a database and later accessed to determine the correlation with the de-embedded permittivity value of an unknown sample, a close correlation indicating the same molecular makeup. The database may be made accessible via electronic means such as a local area network or the Internet to permit users to compare the permittivity values of their unknown samples with the known permittivity values of the database, thereby assisting the user in analyzing the molecular event makeup of their samples.

FIG. 6 illustrates one embodiment of the process 520 in which two permittivity coefficients are defined for the resonator. Initially at 602, a first calibration sample is provided and the sample's complex permittivity κ₁ is measured at one or more frequencies:

∈₁(f)={∈₁′(f₁)−j∈₁″(f₁), ∈₁′(f₂)−j∈₁″(f₂) . . . ∈₁′(fₙ)−j∈₁″(fₙ)} (5)

In a specific embodiment, the first calibration sample is phosphate buffer solution (referred to as “PBS”). In an alternative embodiment, other materials such as DMSO, de-ionized water may be used as the calibration solution. In a specific embodiment, the signal analyzer 110 is a vector network analyzer model no. HP 8722, and the measurement probe is the aforementioned model no. HP 85070C dielectric measurement probe. The dielectric probe includes accompanying software readable by the computer system 105 (a HP Vectra in one embodiment) and is operable to convert the measured s-parameters into permittivity values.

Next at 604, a second calibration sample is provided and the sample's complex permittivity measured at one or more frequencies:

∈₂(f)={∈₂′(f₁)−j∈₂″(f₁), ∈₂′(f₂)−j∈₂″(f₂) . . . ∈₂′(fₙ)−j∈₂″(fₙ)} (6)

The measurement may be made using the aforementioned network analyzer and dielectric measurement probe. The second calibration solution may be contain the solution of the first calibration sample, but at a different concentration, temperature, pH, or other condition. In other embodiments, the second calibration solution may consist of a different material/solution all together. In a specific embodiment, the second calibration solution is 6% ethanol and PBS mixture.

In the preferred embodiment, the measurements of processes 602 and 604 will be made over a range of frequencies f₁ to fₙ which include the critically coupled frequency of the subject resonator. Further preferably, the measurement frequency range will extend over one or more harmonics of the resonant frequency as the resonator will exhibit a similar resonant frequency response at these frequencies as well. The frequency intervals are preferably made small enough so that the change in permittivity across the frequency interval is substantially linear.

Next at 606 and 608, the real and imaginary parts, respectively, of the permittivity values of the first and second calibration samples are subtracted to calculate the permittivity difference quantities, {Δ∈′_{cal}} and {Δ∈″_{cal}}, respectively:

{Δ∈′_{cal}}=∈₂′(f)−∈₁′(f); (7)

{Δ∈″_{cal}}=j∈₂″(f)−j∈₁″(f) (8)

The computer system 105 may be used to calculate the permittivity difference quantities {Δ∈′_{cal}} and {Δ∈″_{cal}} as well as control the supply of the calibration samples to the detector assembly 120, and to control the network analyzer to transmit and receive the incident and reflected test signals.

Permittivity Characterization using Resonant Detector fᵣₑₛ and Q Parameters

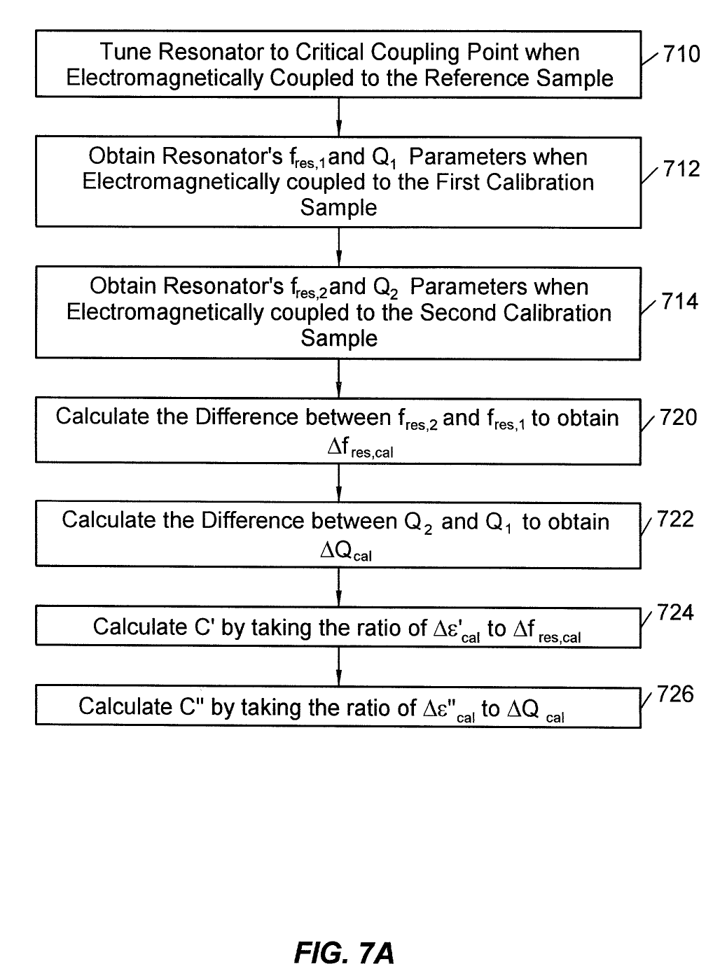

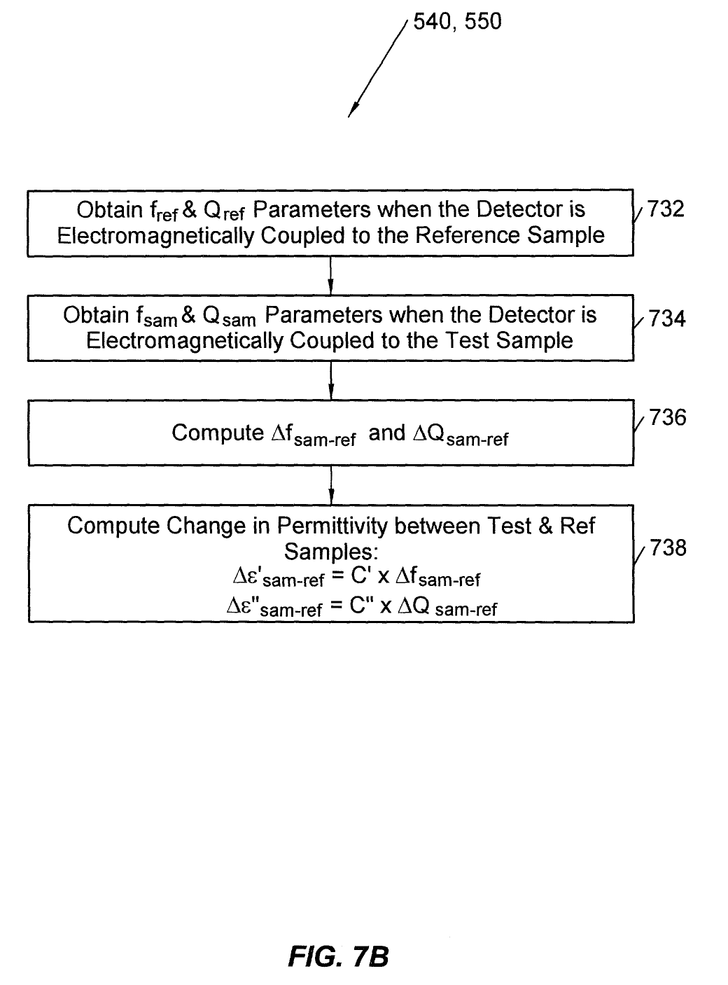

FIG. 7A illustrates one embodiment of process 530 in which the above computed permittivity difference quantities Δ∈′_{cal} and Δ∈″_{cal} are applied to measurement parameters fᵣₑₛ and Q to compute the permittivity coefficients. A resonant detector, such as the coaxial resonator illustrated in FIG. 3A, is used to obtain the fᵣₑₛ and Q parameters, although in other embodiments other resonant structures listed herein may be used in the present invention as well.

The process begins at 710 at which point the resonant detector is tuned to the “critical coupling” point (i.e., point at which the magnitude of the signal response is minimal) when the resonator is electromagnetically coupled to a reference sample, further described below. In the specific embodiment in which a coaxial resonator 330 (FIG. 3A) is used, the tuning process is accomplished by rotating the tuning element 333 clockwise or counter-clockwise, such that the magnitude of the frequency response reaches a minimal point, the corresponding frequency being the resonant frequency (fᵣₑₛ).

The reference sample is the material against which the test sample will be compared during the measurement process. The reference sample may be a sample of the buffer, the native or near-native environment in which the sought molecular event is known to reside, e.g., a sample containing a purified protein, a mixture of proteins, complex systems such as cellular lysates.

Alternatively, the reference sample may be one of the calibration samples, e.g., PBS,DMSO, de-ionized water, or another aqueous environment in which the user seeks to detect/identify the molecular structure or binding event. In this instance, the process of 710 includes supplying the selected calibration sample to the detector region where it is electromagnetically coupled to the resonator, and tuning the resonator to its critically coupled point, as described above.

Next at 712, the first calibration sample is supplied to the resonator's detection region and resonator's output parameters, fᵣₑₛ and Q, are obtained. In a specific embodiment, this process is performed by measuring the input s-parameters of the detector and deriving the resonator's resonant frequency fᵣₑₛ and q-factor Q therefrom, as illustrated in 3B above.

The measurement frequency range will preferably be within the frequency range over which the permittivity difference quantities {Δ∈′_{cal}} and {Δ∈″_{cal}} were measured in processes 602 and 604, above. In a specific embodiment, the resonator exhibits a critically couple frequency near 1.0 GHz when electromagnetically coupled to the first calibration sample and the tested frequency ranges from 0.95 GHz to 1.05 GHz. However, other frequency ranges and/or bandwidths may be used in alternative embodiments. For instance, those skilled in the art of high frequency design will understand that the 1.0 GHz resonant structure will exhibit periodic resonant responses at multiples of the fundamental frequency of 1.0 GHz. In this instance, the test frequency may range over one or more of the harmonic frequencies. A computer-controlled vector network analyzer is used to measure the input s-parameters of the resonator, although other s-parameters may be measured in alternative embodiments.

At process 714, the second calibration sample is provided to the detection region where it is electromagnetically coupled to the detector and the detector's fᵣₑₛ and Q parameters are obtained. The process is preferably performed by measuring the input s-parameters and deriving the resonator's fᵣₑₛ and Q parameters therefrom as described above.

At 720 and 722, the difference in resonant frequencies (Δf_{res,cal}) and Q-factors (ΔQ_{cal}) are computed. The quantity Δf_{res,cal} is correlated to the real part of the calibration sample permittivity and the quantity ΔQ_{cal} is correlated to the imaginary part of the calibration sample's permittivity. As each of the resonant frequency points (f_{res,1} and f_{res,2}) and Q-factors (Q₁ and Q₂) are computed at a single frequency (the critically coupled point for each response), their differences (Δf_{res,cal} and ΔQ_{cal}) will each be a single quantity which does not vary with frequency.

The two permittivity difference quantities Δ∈′_{cal}(fᵢ) and Δ∈″_{cal}(fᵢ) which are obtained at frequency fᵢ nearest to resonant frequencies f_{res,1} and f_{res,2} are selected from {Δ∈′_{cal}} and {Δ∈″_{cal}}, respectively. In one embodiment, the two permittivity difference quantities selected are those at frequency fᵢ which is nearest to the average value of the resonant frequencies f_{res,1} and f_{res,2}.

At 724, the permittivity coefficient C′ is calculated by taking the ratio of Δ∈′_{cal}(fᵢ) to Δf_{res,cal}:

C′=Δ∈′_{cal}(fᵢ)/Δf_{res,cal} (9)

and at 726, the imaginary part of the permittivity coefficient C″ is calculated by taking the ratio of Δ∈″_{cal}(fᵢ) to ΔQ_{cal}:

C″=Δ∈″_{cal}(fᵢ)/ΔQ_{cal} (10)

The signs of the permittivity coefficients C′ and C″ are retained, that is if Δ∈′ (e.g., ∈′₂−∈′₁) is negative and ∈fᵣₑₛ (e.g., f_{res,2}−f_{res,1}) is positive, the permittivity coefficient C′ will be negative, indicating a negative change in the permittivity with increasing frequency (f_{res,2} is higher in frequency than f_{res,1} in the foregoing example).

The processes illustrated in FIG. 7A may be repeated whenever a change occurs to the test station (e.g., when the resonator is changed, when the network analyzer or one of its components are changed, when the flow tube is replaced, when the critical coupling point shifts or is re-adjusted, etc.), or after a predetermined period in order to maintain an accurate calibration of the test station. The process of FIG. 6 will not require frequent repetition if the prepared calibration samples are maintained at their specific concentrations.

FIG. 7B illustrates one embodiment of the processes 540 and 550 in which parameters fᵣₑₛ and Q are applied to the permittivity coefficients C′ and C″ to compute the test sample permittivity. The process begins at 732 at which point the resonator's output parameters f_{ref} and Q_{ref} are obtained when the reference sample is supplied to the detection region and electromagnetically coupled to the resonant detector. In one embodiment, the computer-controlled network analyzer 110 is programmed to measure the resonator's input s-parameter response over a predefined frequency range and to compute the resonant frequency and Q factor of the resonant detector.

At 734, the resonator's output parameters fₛₐₘ and Q_{sam.} are obtained when the test sample is supplied to the detection region and electromagnetically coupled to the resonant detector. This process may be similarly performed using the computer-controlled network analyzer 110 as described above.

Next at 736, difference quantities Δf_{sam-ref} and ΔQ_{sam-ref} are computed by subtracting f_{ref} from fₛₐₘ, and Q_{ref} from Qₛₐₘ, respectively. Next at 738 (a specific embodiment of process 550), the permittivity value of the test sample, which in the illustrated embodiment is a change in the permittivity between the test and reference samples, is computed applying the real and imaginary permittivity coefficients C′ and C″ to the difference quantities Δf_{res,sam-ref}, ΔQ_{sam-ref}:

Δ∈′_{sam-ref}=C′*Δf_{sam-ref}; (11)

and

Δ∈″_{sam-ref}=C″*ΔQ_{sam-ref} (12)

From an inspection of eqs. (11) and (12), it will be seen that the permittivity of the test sample itself ∈′ₛₐₘ and ∈″ₛₐₘ can be computed by adding the permittivity of the reference sample ∈′_{ref} and ∈″_{ref} to the difference quantities Δ∈′_{sam-ref} and Δ∈″_{sam-ref}. Alternatively, the permittivity of the reference sample ∈′_{ref} and ∈″_{ref} may be computed by subtracting the difference quantities Δ∈′_{sam-ref} and Δ∈″_{sam-ref} from the test sample permittivity ∈′ₛₐₘ and ∈″ₛₐₘ.

Permittivity Characterization Using Resistance and Reactance Parameters

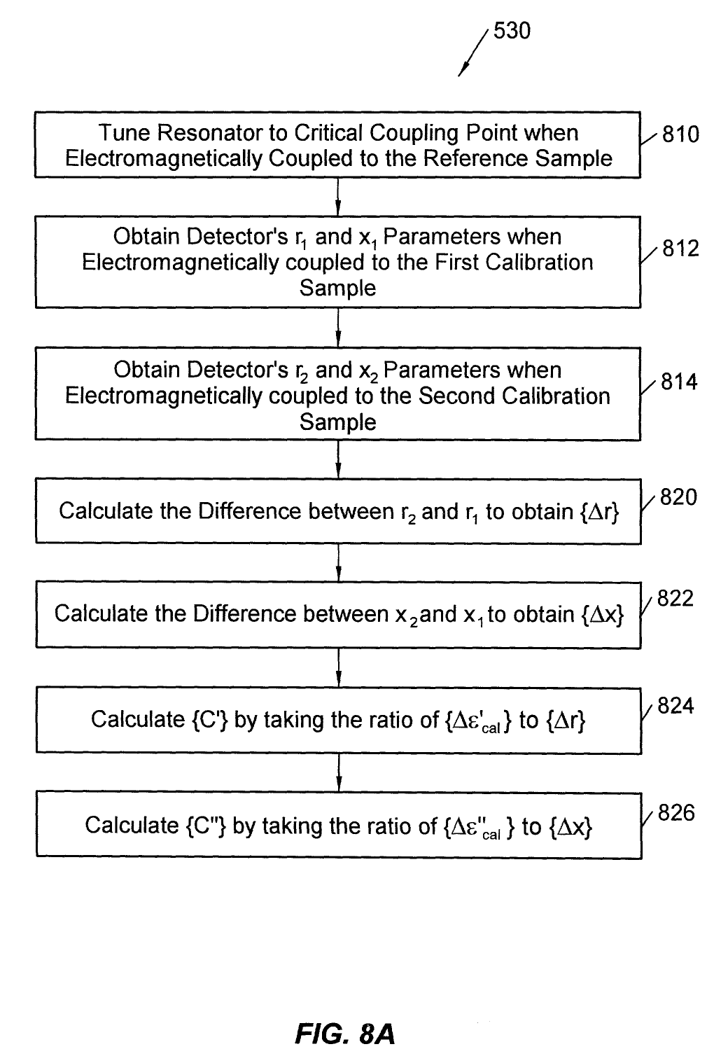

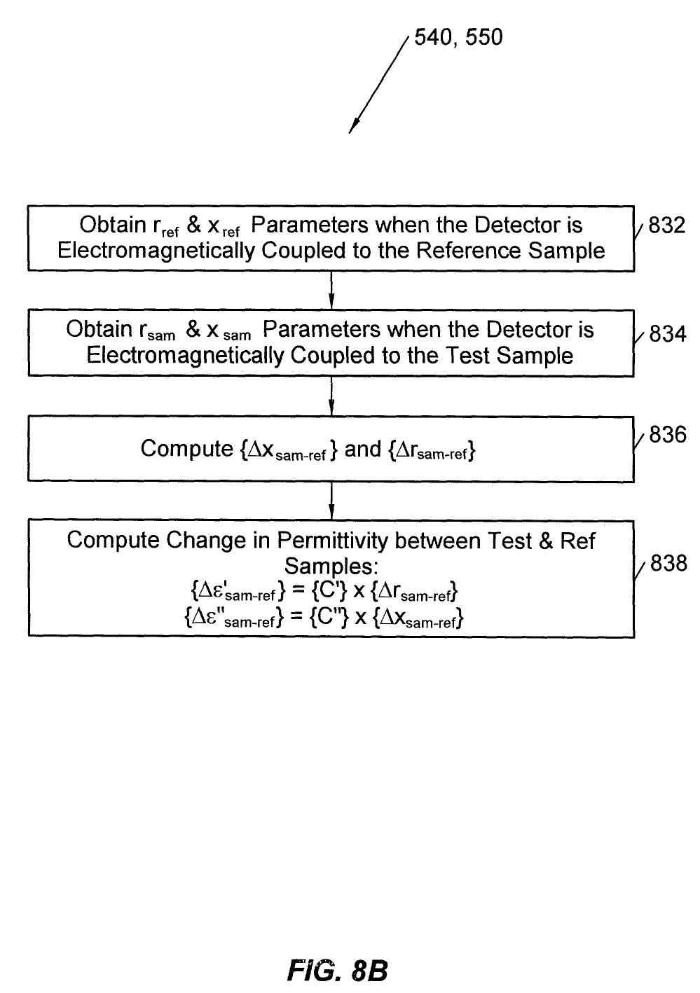

FIG. 8A illustrates a second embodiment of process 530 in which the above computed permittivity difference quantities Δ∈′_{cal} and Δ∈″_{cal} are applied to the resistance (r) and reactance (x) parameters to compute the permittivity coefficients. This process may be used when a resonant structure is employed or alternatively when a broadband detector is used.

If a resonant detector is used, the process begins at 810 where the resonator is tuned to critical coupling point when electromagnetically coupled to the reference sample. When the coaxial resonator 330 (FIG. 3A) is implemented, tuning to the critical coupled point is performed by rotating the tuning element 333 clockwise or counter-clockwise until a minimum amplitude point of the input s-parameter (S₁₁) is reached. The reference sample can be any of the samples listed herein. If a non-resonant detector is used, this process is omitted.

At 812, the output parameters, r₁ and x₁, of the detector are obtained when electromagnetically coupled to the first calibration sample. In a specific embodiment, this process is performed by measuring the input s-parameters of the detector and deriving the resistance and reactance terms at each measurement frequency, as illustrated above.

In one embodiment of this process in which a resonant structure such as the coaxial resonator illustrated in FIG. 3A is used, the detector's input s-parameters are measured over a relative narrow frequency range, for instance, 1 GHz +/−500 KHz. In alternative embodiment in which a non-resonant detector is employed, the detector's input s-parameters are measured over a relative wide frequency range, for instance, 10 MHz to 3 GHz, or 45 MHz to 20 GHz for example. Those of skill in the art will appreciate that other s-parameter responses (or g-, h-, y, or z-parameter) over different frequency ranges may be used in alternative embodiments under the present invention.

A set of resistance terms and reactance terms is obtained as the following (“f” denotes the measurement frequency):

r₁(f)={r₁(f₁), r₁(f₂) . . . r₁(fᵢ)} (13)

x₁(f)={x₁(f₁), x₁(f₂) . . . x₁(fᵢ)} (14)

The set or resistances and reactances may consist of a single term or multiple terms, depending upon which of the measurement frequencies are used. For example, when a resonant detector is used, only one of the measurement frequencies, the frequency nearest the resonant frequency of the detector, may be used. The point provides high detector sensitivity and may be the only frequency point or one or a few points used. Alternatively, the set may include a large number of terms, corresponding to an interrogation over a large number of frequency points. A large number of points may be used, for instance, when a broadband detector is used to obtain a broadband response over a wide spectrum.

At process 814, the second calibration sample is provided to the detection region where it is electromagnetically coupled to the detector and the detector's output parameters are obtained:

r₂(fᵢ)={r₂(f₁), r₂(f₂) . . . r₂(fₙ)} (15)

x₂(fᵢ)={x₂(f₁), x₂(f₂) . . . x₂(fₙ)} (16)

At 820 and 822, the difference in resistance and reactance components and [r₂(fᵢ)−r₁(fᵢ)] and [x₂(fᵢ)−x₁(fᵢ)], respectively, for all fᵢ, and the differences are compiled within sets {Δr} and {Δx}. Each term within the set {Δr} represents the real part of the detector' impedance at a particular frequency and each term within the set {Δx} represents the imaginary part of the detector's impedance at a particular frequency.

In an embodiment in which two or more resistance and/or reactance parameters are obtained for processes 812-818, e.g., a broadband detector, the sets {Δr} and {Δx} will include two or more difference terms, each term at frequency fᵢ. In an embodiment in which only one resistance and/or reactance parameter is obtained from processes 812-818, e.g., in the resonant detector embodiment, the sets {Δr} and {Δx} will include only one term representing the difference in resistance and reactance, respectively.

At 824, the set of real parts of the permittivity coefficient {C′} is calculated by taking the ratio of {Δ∈′_{cal}} to {Δr}:

{C′}={Δ∈′_{cal}}/{Δr}; (17)

and at 826, the set of imaginary parts of the permittivity coefficient {C″} is calculated by taking the ratio of {Δ∈″_{cal}} to {Δx}:

{C″}={Δ∈″_{cal}}/{Δx} (18)

As illustrated, the set of real coefficients {C′} comprises one or more terms C(fᵢ) consisting of the quotient Δ∈′_{cal}(fᵢ)/Δr(fᵢ), whereby Δ∈′_{cal}(fᵢ) and Δr(fᵢ) are corresponding terms nearest to frequency fᵢ. The set of imaginary coefficients {C″} is similarly computed. In an embodiment in which a resonant detector is employed, the sets of real and imaginary coefficients may consists only of a single term, Δr(fᵣₑₛ) and Δx(fᵣₑₛ), representing the resistance and reactance at the resonant frequency point fᵣₑₛ. In this instance the Δ∈′_{cal} and Δ∈″_{cal} terms are chosen such that they are the closest terms to the frequency point fᵣₑₛ.

FIG. 8B illustrates an embodiment of the processes 540 and 550 in which parameters r and x are applied to the permittivity coefficients C′ and C″ to compute the test sample permittivity. The process begins at 832 at which point the detector's output parameters r_{ref} and x_{ref} are obtained when the reference sample is supplied to the detection region and electromagnetically coupled to the detector. In one embodiment, the computer-controlled network analyzer 110 is programmed to measure the detector's input s-parameter response over a predefined frequency range and to compute the detector's resistance and reactance as a function of frequency.

At 834, the detector's output parameters rₛₐₘ and xₛₐₘ are obtained when the test sample is supplied to the detection region and electromagnetically coupled to the resonant detector. This process may be similarly performed using the computer-controlled network analyzer 110 as described above.

Next at 836, difference quantities Δr_{sam-ref} and Δx_{sam-ref} are computed by subtracting r_{ref} from rₛₐₘ and x_{ref} from xₛₐₘ. Next at 838 (a specific embodiment of process 550), the permittivity value of the test sample, which in the illustrated embodiment is a change in the permittivity between the test and reference samples, is computed applying the real and imaginary permittivity coefficients C′ and C″ to the difference quantities Δr_{res,sam-ref}, Δx_{sam-ref}:

{Δ∈′_{sam-ref}}={C′}*{Δr_{sam-ref}} (18)

{Δ∈″_{sam-ref}}={C″}*{Δx_{sam-ref}} (19)

The set {Δ∈′_{sam-ref}} represents the real part of the test permittivity relative to the reference sample and consists of one or more terms Δr_{sam-ref}(fᵢ)*C′(fᵢ), whereby Δr_{sam-ref}(fᵢ) and C(fᵢ) are corresponding terms nearest to frequency fᵢ. The set {Δ∈″_{sam-ref}} represents the imaginary part of the test permittivity relative to the reference sample and is similarly computed.

Permittivity Characterization Using I and Q Components of S-parameters

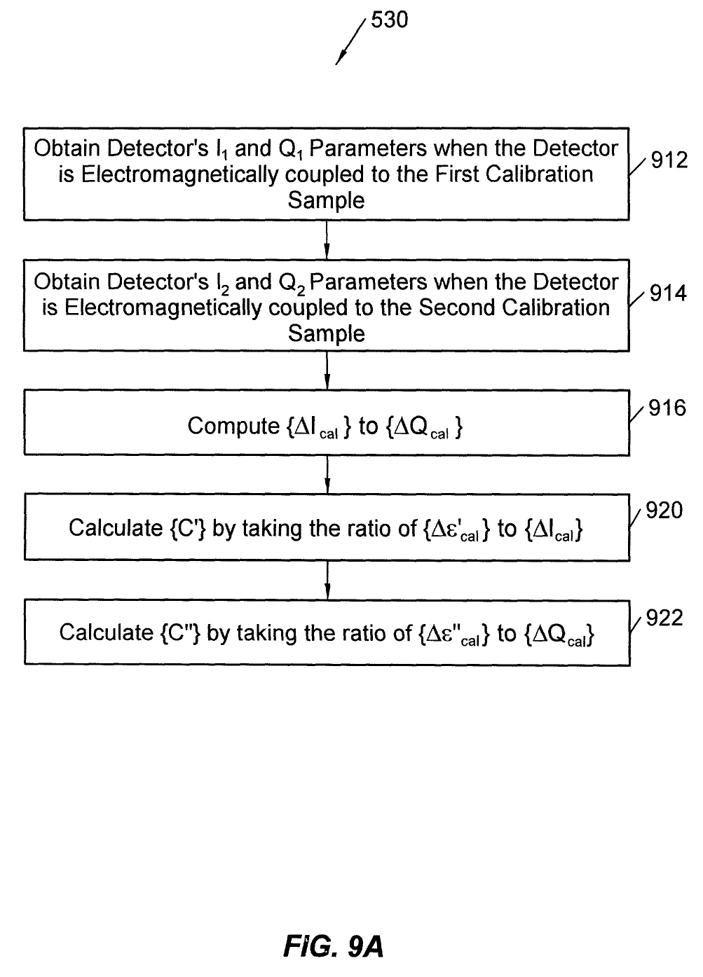

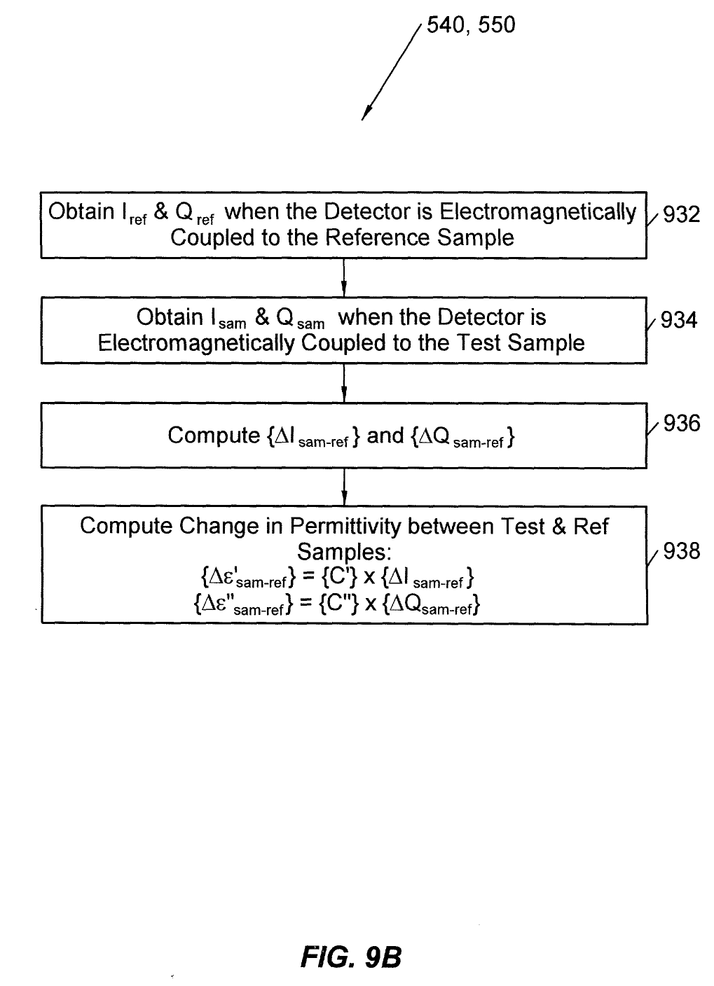

FIG. 9A illustrates an embodiment of process 530 in which the above computed permittivity difference quantities Δ∈′_{cal} and Δ∈″_{cal} are applied to real (I) and imaginary (Q) components of an s-parameter measurement to compute the permittivity coefficients. As known to those skilled in the art of high frequency circuit design and analysis, s-parameters are typically measured in terms of an I or in-phase component, and a Q or quadrature phase component, which are directly measurable by a signal analyzer such as a network analyzer.

At 912, the first calibration sample is supplied to the detector and the detector's input s-parameters are measured at one or more frequencies. The measured s-parameters will be in the form of real and imaginary components, I and Q, respectively:

I₁(f)={I₁(f₁), I₁(f₂) . . . I₁(fᵢ)}; (20)

x₁(f)={Q₁(f₁), Q₁(f₂) . . . Q₁(fᵢ)}; (21)

Next at 914, the second calibration sample is supplied to the detector and the detector's input s-parameters are measured at one or more frequencies:

I₂(f)={I₂(f₁), I₂(f₂) . . . I₂(fᵢ)}; (22)

Q₂(f)={Q₂(f₁), Q₂(f₂) . . . Q₂(fᵢ)}; (23)

At 916, the difference in I and Q signal components are computed as [I₂(fᵢ)−I₁(fᵢ)] and [Q₂(fᵢ)−Q₁(fᵢ)], respectively, for all fᵢ, and the differences are compiled within sets {ΔI_{cal}} and {ΔQ_{cal}}. Each term within the set {ΔI_{cal}} represents the real part of the detector' s-parameter measurement at a particular frequency and each term within the set {ΔQ_{cal}} represents the imaginary part of the detector's measured s-parameter at a particular frequency.

At 920, the set of real parts of the permittivity coefficient {C′} is calculated by taking the ratio of {Δ∈′_{cal}} to {ΔI_{cal}}:

{C′}={Δ∈′_{cal}}/{ΔI_{cal}} (24)

and at 922, the imaginary part of the permittivity coefficient C″ is calculated by taking the ratio of Δ∈″_{cal} to ΔQ_{cal}:

{C″}={Δ∈″_{cal}}/{ΔQ_{cal}} (25)

The set of real coefficients {C′} comprises one or more terms C(fᵢ) consisting of the quotient Δ∈′_{cal}(fᵢ)/ΔI_{cal}(fᵢ), whereby Δ∈′_{cal}(fᵢ) and ΔI_{cal}(fᵢ) are corresponding terms nearest to frequency fᵢ. The set of imaginary coefficients {C″} is similarly computed. In an embodiment in which a resonant detector is employed, the sets of real and imaginary coefficients may consists only of a single term, ΔI_{cal}(fᵣₑₛ) and ΔQ_{cal}(fᵣₑₛ), representing the in-phase (I) and quadrature phase (Q) signal components at the resonant frequency point fᵣₑₛ. In this instance the Δ∈′_{cal}(fᵢ) and Δ∈″_{cal}(fᵢ) terms are chosen such that they are the closest terms to the frequency point fᵣₑₛ.

The processes of FIG. 9A may be repeated whenever a change occurs to the test station (e.g., when the resonant probe is changed, when the network analyzer or one of its components are changed, when the flow tube is replaced, when the critical coupling point shifts or is re-adjusted, etc.), or after a predetermined period in order to maintain an accurate calibration of the test station.

FIG. 9B illustrates an embodiment of the processes 540 and 550 in which the I and Q components of the measured s-parameters are applied to the permittivity coefficients C′ and C″ to compute the test sample permittivity. The process begins at 932 at which point the detector's output parameters I_{ref} and Q_{ref} are obtained when the reference sample is supplied to the detection region and electromagnetically coupled to the resonant detector. In one embodiment, the computer-controlled network analyzer 110 is programmed to measure the I and Q components of the detector's input s-parameters over a predefined frequency range.

At 934, the detector's output parameters Iₛₐₘ and Qₛₐₘ are obtained when the test sample is supplied to the detection region and electromagnetically coupled to the resonant detector. This process may be similarly performed using the computer-controlled network analyzer 110 as described above.

Next at 936, difference quantities ΔI_{sam-ref} and ΔQ_{sam-ref} are computed by subtracting I_{ref} from Iₛₐₘ and Q_{ref} from Qₛₐₘ. Next at 938 (a specific embodiment of process 550), the permittivity value of the test sample, which in the illustrated embodiment is a change in the permittivity between the test and reference samples, is computed applying the real and imaginary permittivity coefficients C′ and C″ to the difference quantities ΔI_{res,sam-ref}, ΔQ_{sam-ref}:

{Δ∈′_{sam-ref}}={C′}*{ΔI_{sam-ref}} (26)

{Δ∈″_{sam-ref}}={C″}*{ΔQ_{sam-ref}} (27)

The set {Δ∈′_{sam-ref}} represents the real part of the test permittivity relative to the reference sample and consists of one or more terms ΔI_{sam-ref}(fᵢ)*C′(fᵢ), whereby ΔI_{sam-ref}(fᵢ) and C(fᵢ) are corresponding terms nearest to frequency fᵢ. The set {Δ∈″_{sam-ref}} represents the imaginary part of the test permittivity relative to the reference sample and is similarly computed.

Permittivity Characterization Using Bilinear Calibration

Bilinear calibration techniques have been applied successfully to broadband, open-ended coaxial probe systems. The premise behind the technique is the assumption that a bilinear relationship of the form:

$${Γ}_{t} = \frac{Aϵ + B}{Cϵ + 1} \qquad \text{eq. (28)}$$

exists between the true reflection coefficient Γₜ and the complex permittivity ∈. A, B, and C are complex constants. The true reflection coefficient is related to the measured reflection coefficient Γₘ by the expression:

$${Γ}_{m} = {e}_{d} + \frac{{e}_{r} {Γ}_{t}}{1 - {e}_{{sΓ}_{t}}} \qquad \text{eq. (29)}$$

where e_{d}, eᵣ, and eₛ are error terms corresponding to directivity, frequency response, and source match. The measured reflection coefficient can be further reduced to:

$${Γ}_{m} = \frac{\tilde{A} ϵ + \tilde{B}}{\tilde{C} ϵ + 1} \qquad \text{eq. (31)}$$

where Ã, \tilde{B}, and \tilde{C} are complex constants computed at each sampled frequency. These complex coefficients can be determined by measuring the reflection coefficients of three fluids with known permittivities. Equation 30 can be manipulated to solve for the complex permittivity as a function of the computed reflection coefficients:

∈=[Γₘ−B]/[A−C*Γₘ] eq. (31)

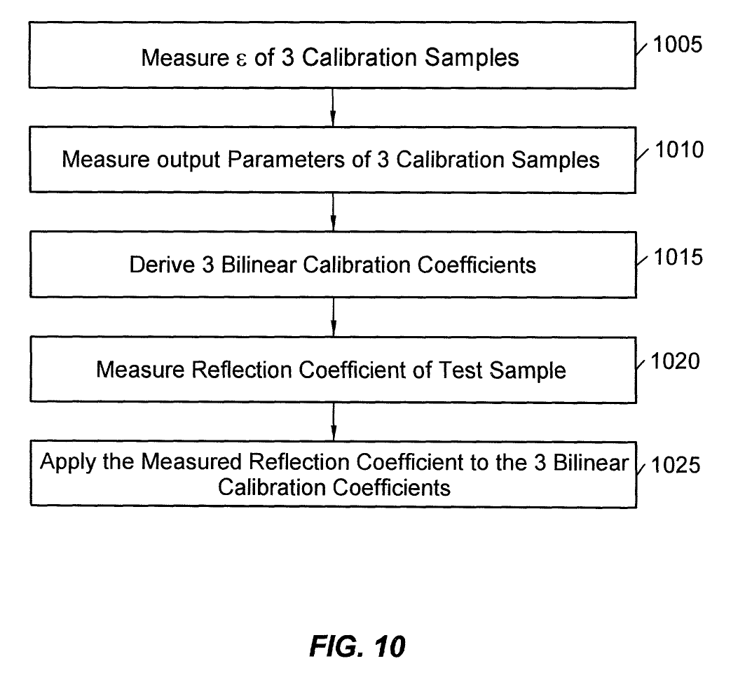

FIG. 10 illustrates a method for characterizing the permittivity of a molecular event in a test sample using the aforementioned bilinear calibration technique. Initially at 1005, the permittivity of three calibration samples is measured. In one embodiment, the three following fluids are chosen as calibration samples: buffer such as PBS, a diluted version of the buffer, and the buffer plus a fixed volume fraction of polystyrene beads. The diluted buffer operates to set the high permittivity range, and the solution with beads sets the low end. The permittivity measurement may be performed using a commercially available dielectric probe such as the model number 85070 manufactured by Agilent Technologies (Palo Alto, Calif.).

Subsequently at 1010, the output parameters of the three calibration samples are measured. In a specific embodiment, the output parameter is the input reflection coefficient of the three calibration samples measured over frequency. Next at 1015, the three calibration coefficients are derived using the eq. (30) above. At 1020, the input reflection coefficient of the test sample is measured over frequency. In the preferred embodiment, the test sample is measured over the same frequency range and at the same frequency points as the three calibration samples. At 1025, the three derived calibration coefficients are applied to the measured reflection coefficient of the test sample, and a measured permittivity is computed as per eq. (31). The computed permittivity may be either an absolute value figure or made relative to a reference sample if the reflection coefficient is made relative to the reflection coefficient of a reference sample.

Sample Temperature Computation Process

The aforementioned permittivity values have been described as a function of frequency, and without any dependency of the sample's temperature. Accordingly, the foregoing permittivity values are henceforth referred to as “temperature-independent” permittivities as their computation are not a function of and are not informed by the temperature of the sample. It is known, however that a sample's temperature may also impact its permittivity at one or more frequencies.

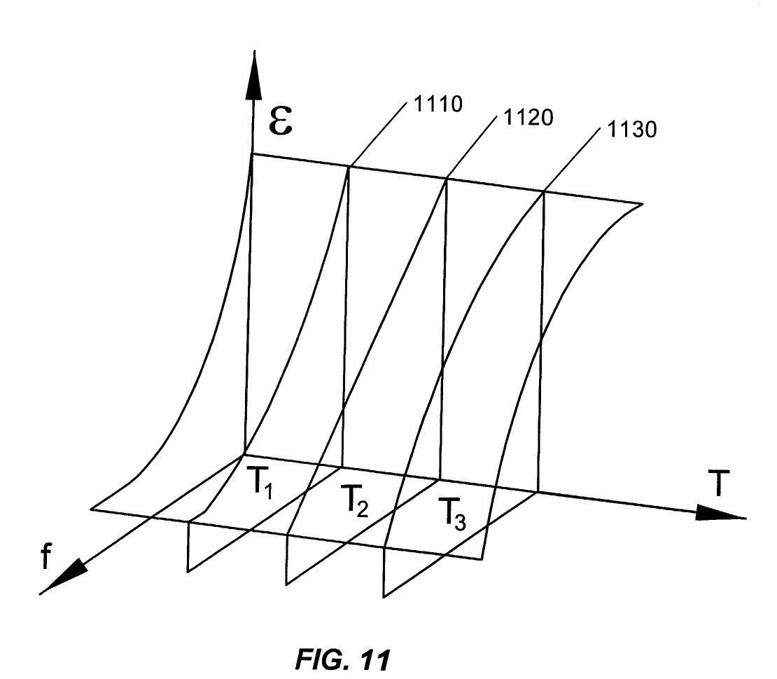

FIG. 11 illustrates a graph showing a sample's permittivity varying with frequency and temperature. As can be seen, the sample's dispersive response, i.e., the permittivity-v-frequency response forms different surface contours at different temperatures. At temperature T₁ the permittivity-v-frequency response 1110 resembles a concave contour, at temperature T₂, the response 1120 has a generally linear contour, and at temperature T₃ the response 1130 exhibits a generally convex contour. The illustrated temperature variation is exemplary and each sample is expected to have a unique dispersive response versus temperature, although samples within the same class will be more closely correlated than those outside of their class. Accordingly, computing the sample's permittivity as a function of temperature provides another basis to identify (or distinguish) two or more samples or group related samples.

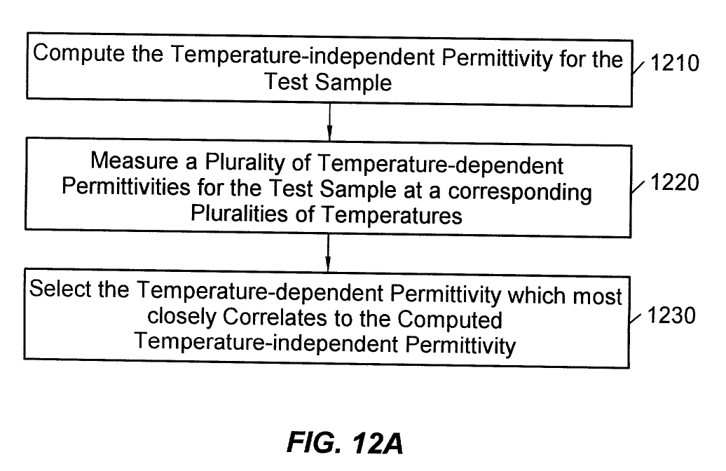

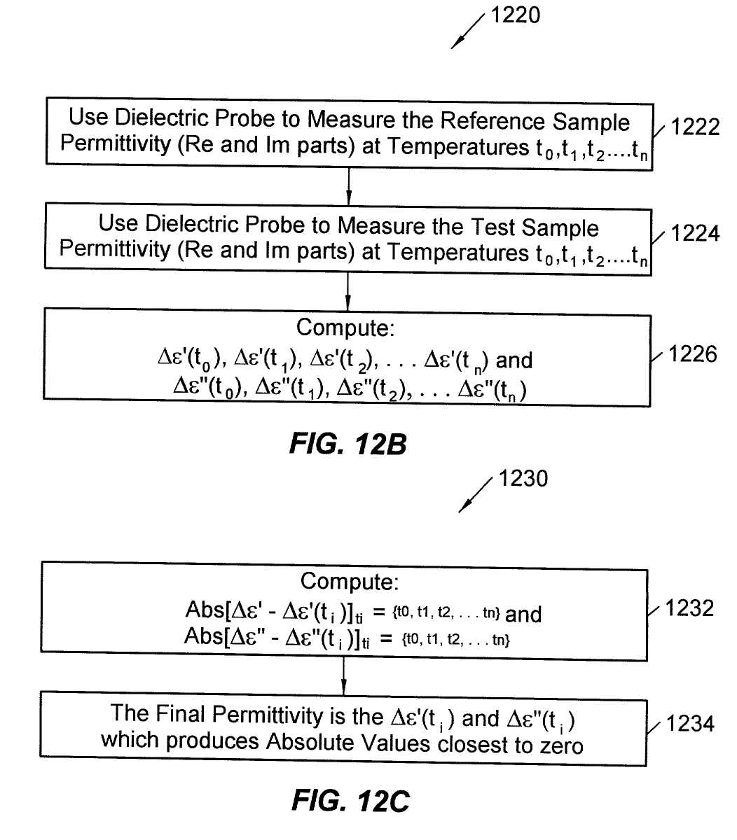

FIG. 12A illustrates one embodiment of the process by which the temperature-dependent permittivity of a sample is computed in accordance with the present invention. Initially at 1210, the temperature-independent permittivity of the test sample is determined. As defined above, the temperature-independent permittivity does not include a temperature dependency, and its computation is not informed by the temperature of the sample. One embodiment of determining the temperature-independent permittivity is illustrated in FIG. 5 above. In a specific embodiment, the temperature-independent difference quantities Δ∈′_{sam-ref} and Δ∈″_{sam-ref} are determined using the processes illustrated above.

Once the temperature-independent permittivity of the test sample is computed, at 1220 the permittivity of the test sample is measured at x different temperatures T₁, T₂, . . . Tₓ and the resulting permittivity measurements are compiled in a set {∈(T₁), ∈(T₂), ∈(T₃), . . . ∈(Tₓ)}. These permittivities {∈(T₁), ∈(T₂), ∈(T₃), . . . ∈(Tₓ)} are henceforth referred to as “temperature-dependent” permittivities as their values are dependent upon the temperature of the sample. In a specific embodiment, the temperature-dependent permittivities of the test and reference samples are each measured at different temperatures T₁, T₂, T₃, . . . Tₓ. The difference therebetween is computed at each temperature, and a set of temperature-dependent difference permittivities {Δ∈(T₁), Δ∈(T₂), Δ∈(T₃), . . . Δ∈(Tₓ)} is compiled. An embodiment of this process is further illustrated in FIG. 12B and described below.

Subsequently at 1230, the temperature-dependent permittivity ∈(Tᵢ) which is most closely correlated to the temperature-independent permittivity is selected from the set x as the permittivity of the test sample. In a specific embodiment, the selected temperature-dependent permittivity is the temperature-dependent difference permittivity Δ∈(Tᵢ) which is most closely correlated to the temperature-independent difference permittivity Δ∈_{sam-ref}. An embodiment of this process is further illustrated in FIG. 12C below.

FIG. 12B illustrates one embodiment of the process 1220 in which the test sample permittivity is measured at a plurality of distinct temperatures T₁, T₂, T₃, . . . Tₓ. Initially at 1222, a dielectric probe such as model no. HP85070 is used to measure the permittivity (real and imaginary parts) of a reference sample at temperatures T₁, T₂, T₃, . . . Tₓ. Two sets of temperature-dependent permittivity values are generated corresponding to the real and imaginary parts of the measured permittivity:

Re={∈′_{ref}(T₁), ∈′_{ref}(T₂), ∈′_{ref}(T₃), . . . ∈′_{ref}(Tₙ)} (28)

Im={∈″_{ref}(T₁), ∈″_{ref}(T₂), ∈″_{ref}(T₃), . . . ∈″_{ref}(Tₙ)} (29)

Next at 1224, the dielectric probe is used measure the real and imaginary parts of the test sample permittivity at temperatures T₁, T₂, T₃, . . . Tₓ. Two sets of temperature-dependent permittivity values are generated corresponding to the real and imaginary parts of the measured permittivity:

Re={∈′ₛₐₘ(T₁), ∈′ₛₐₘ(T₂), ∈′ₛₐₘ(T₃), . . . ∈′ₛₐₘ(Tₙ)} (30)

Im={∈″ₛₐₘ(T₁), ∈″ₛₐₘ(T₂), ∈″ₛₐₘ(T₃), . . . ∈″ₛₐₘ(Tₙ)} (31)

At 1226, at each temperature the test sample temperature-dependent permittivity value is subtracted from the reference sample temperature-dependent permittivity value, resulting in the following difference permittivity values in real and imaginary terms, respectively:

Re={Δ∈′_{sam-ref}(T₁), Δ∈′_{sam-ref}(T₂), Δ∈′_{sam-ref}(T₃), . . . Δ∈′_{sam-ref}(Tₙ)} (32)

Im={Δ∈″_{sam-ref}(T₁), Δ∈″_{sam-ref}(T₂), Δ∈″_{sam-ref}(T₃), . . . Δ∈″_{sam-ref}(Tₙ)} (33)

FIG. 12C illustrates a process for selecting the final permittivity for the test sample. At 1232, the absolute value of the difference between the temperature-dependent and each of the temperature-independent difference permittivity values (real and imaginary parts) are computed:

Abs[Δ∈′_{sam-ref}(Tᵢ)−Δ∈′_{sam-ref}] for Tᵢ={T₀, T₁, T₂, . . . Tₙ} (34)

Abs[Δ∈″_{sam-ref}(Tᵢ)−Δ∈″_{sam-ref}] for Tᵢ={T₀, T₁, T₂, . . . Tₙ} (35)

At 1234, the temperature-dependent difference permittivity values Δ∈′_{sam-ref}(Tᵢ) and Δ∈″_{sam-ref}(Tᵢ) producing the result closest to zero are selected from their corresponding sets. The values Δ∈′_{sam-ref}(Tᵢ) and Δ∈″_{sam-ref}(Tᵢ) represent the temperature-dependent difference in permittivity between the test and reference samples previously computed Δ∈′_{sam-ref} and further provides the temperature of the reference and test samples at which that measurement Δ∈′_{sam-ref} was made.

The following commonly-owned, co-pending patent applications, as well as all publications and patent documents recited in this application are incorporated by reference in their entirety for all purposes to the same extent as if each individual publication and patent document was so individually denoted:

Ser. No. 09/243,194, entitled: “Method and Apparatus for Detecting Molecular Binding Events,” filed Feb. 1, 1999 (Atty Docket No 19501-000200);

Ser. No. 09/365,578, entitled “Method and Apparatus for Detecting Molecular Binding Events,” filed Aug. 2, 1999 (Atty Docket No. 19501-000210);

Ser. No. 09/365,978, entitled: “Test Systems and Sensors For Detecting Molecular Binding Events,” filed Aug. 2, 1999 (Atty Docket No. 19501-000500); and

Ser. No. 09/687,456, entitled “System and Method for Detecting Molecular Events in a Test Sample,” filed Oct. 13, 2000 (Atty Docket No. 12.0 US).

What is claimed is:

1. A method for characterizing the permittivity of a molecular event, the method comprising:
obtaining a first permittivity value for a test sample, the test sample comprising:
a known molecular event; and
a buffer;
obtaining a second permittivity value for a reference sample, the reference sample containing the buffer; and
computing the difference between the first and second permittivity values, wherein the computed difference represents the permittivity of the known molecular event.

2. A method for detecting the presence or absence of a known molecular event in a test sample, the method comprising:
obtaining a first permittivity value for a reference sample, the reference sample known to either (1) contain the known molecular event, or (2) exclude the known molecular event;
obtaining a second permittivity value for a test sample suspected of containing the known molecular event;
computing the difference between the first and second permittivity values, wherein the similarity or difference between
computing the difference between the first and second permittivity values, wherein the computed difference represents the permittivity of the known molecular event.

3. A method for determining the relative difference between the permittivity of a test sample and the permittivity of a reference sample, the method comprising:
providing a detector configured to supply output parameters when the detector is electromagnetically coupled to a supplied sample;
defining one or more permittivity coefficients for the detector;
obtaining a first output parameter from the detector when the detector is electromagnetically coupled to a reference sample;
obtaining a second output parameter from the detector when the detector is electromagnetically coupled to the test sample;
applying the difference between the first and second output parameters to the one or more permittivity coefficients to compute the relative difference in permittivity between the test sample and reference sample.
